(19) United States
(12) Patent Application Publication
(10) Pub. No.: US 2006/0210604 A1
(43) Pub. Date: Sep. 21, 2006

(54) Ocular delivery of polymeric delivery formulations

(76) Inventors: Eric Dadey, Fort Collins, CO (US); Christopher M. Lindemann, Loveland, CO (US); Stephen L. Warren, Fort Collins, CO (US); Richard L. Norton, Ft. Collins, CO (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/244,438

(22) Filed: Oct. 4, 2005

Related U.S. Application Data
(60) Provisional application No. 60/615,727, filed on Oct. 4, 2004.
(60) Provisional application No. 60/628,630.
(60) Provisional application No. 60/629,133, filed on Nov. 18, 2004.

(51) Int. Cl. A61F 2/00

(52) U.S. Cl. 424/427; 424/428

(57) ABSTRACT

The present invention provides a flowable composition suitable for use as a controlled release implant. The flowable composition can be administered into the ocular region of a mammal. The composition includes: (a) a biodegradable, biocompatible thermoplastic polymer that is at least substantially insoluble in aqueous medium, water or body fluid; (b) a biological agent, a metabolite thereof, a biological agently acceptable salt thereof, or a prodrug thereof; and (c) a biocompatible organic liquid, at standard temperature and pressure, in which the thermoplastic polymer is soluble. The present invention also provides methods of medical treatment that include administering the flowable composition into the ocular region of a mammal.

### BACKGROUND OF THE INVENTION

The treatment of the eye for disease and/or wounds requires that the particular biological agent be maintained at the site of treatment for an effective period of time. Given the tendency of natural bodily fluids such as tears to rapidly wash away topically applied biological agent components, local ocular therapy or use of the conjunctiva as a route for systemic administration has been problematic.

The use of ocular inserts for the delivery of drugs locally has been described for over 30 years (see, e.g., Ness, U.S. Pat. No. 3,416,530 and Cheng, U.S. Pat. No. 4,053,580). These original inserts included materials that were not soluble or bioerodible in tear fluids.

Other disclosures describe ocular delivery inserts that dispense drugs over a period of time and eventually are completely eroded, but none of these references have suitable bioadhesive capability. See, e.g., Whitaker, et al. (U.S. Pat. No. 3,963,025); Miyata, et al. (U.S. Pat. No. 4,164,559); Cohen, et al. (U.S. Pat. No. 4,179,497); Heller, et al. (U.S. Pat. Nos. 4,346,709 and 4,249,431); Darougar, et al. (U.S. Pat. No. 6,264,971); Wong, et al. (U.S. Pat. No. 6,331,313) and Masters (U.S. Pat. No. 6,342,250).

Flowable solutions of bioadhesive polymer mixtures have also been described to increase the residence time of eyedrops (Bowman et al., U.S. Pat. No. 6,372,245 and Chiou, U.S. Pat. No. 5,283,236). These solutions, however, do not maintain intimate contact with the conjunctiva to achieve rapid onset of therapeutic effects.

The eye is an anatomically complex organ that offers unique challenges and advantages for both the local and systemic delivery of biological agents. The surface epithelial tissues of the eye, the conjunctiva or cornea, are wet tissues constantly bathed with tears. This usually steady flow of moisture drains into the nasal lacrimal ducts at the medial canthus.

The eye's first response to a foreign object is increased tearing, which either washes the foreign matter out of the eye, or for biological agents in eye drops, washes the drug into the sinuses. The inner surface of the eyelid, or palpebral conjunctiva, is a moist, highly vascularized tissue. While the majority of biological agents in an eye drop drains from the sinuses into the back of the throat, some of the biological agent will be taken into the vasculature and become systemic and some will penetrate through the bulbar conjunctiva to the anterior chamber of the eye.

While transport into the systemic circulation is rapid, the efficiency of delivery from eye drops is low, and there is always potential for toxicity because topically applied drugs can readily gain access to the anterior segment of the eye.

Several references describe flowable compositions suitable for use as a controlled release implant, sustained release delivery systems for use as biodegradable and bioerodible implants; wherein the flowable compositions and sustained release delivery systems include: (a) a biodegradable, biocompatible polymer; (b) a biological agent; and (c) a biocompatible organic liquid; and wherein the resulting implants that are formed in situ include: (a) a biodegradable, biocompatible polymer and (b) a biological agent. See, e.g., U.S. Pat. Nos. 6,565,874; 6,528,080; RE37,950; 6,461,631; 6,395,293; 6,355,657; 6,261,583; 6,143,314; 5,990,194; 5,945,115; 5,792,469; 5,780,044; 5,759,563; 5,744,153; 5,739,176; 5,736,152; 5,733,950; 5,702,716; 5,681,873; 5,599,552; 5,487,897; 5,340,849; 5,324,519; 5,278,202; and 5,278,201. These references do dot describe such flowable compositions suitable for use as a controlled release implant wherein the compositions are suitable for ocular delivery.

Accordingly, what is needed is a biological agent carrier for ocular (e.g., transconjunctival or transcorneal) delivery of biological agents for either systemic or local therapy, over variable lengths of time, e.g., delivery occurring for minutes or hours.

### SUMMARY OF THE INVENTION

The formulation of the present invention offers a number of distinct advantages over other parenteral sustained-release delivery systems. For example, microspheres must be manufactured using aseptic processes that may include the use of halogenated solvents. Furthermore, the drug to microsphere ratio is controlled by the encapsulation efficiency, a process that can result in the irretrievable loss of 25 to 50% of the API during the manufacture of the drug product. In comparison, the formulation of the present invention is composed of biocompatible ingredients and is prepared by dissolving the appropriate biodegradable polymer in a biocompatible solvent. Unlike microspheres, the formulation of the present invention can be terminally sterilized using conventional techniques, including gamma irradiation. The unique manufacturing process and proprietary product configuration essentially eliminates the loss of drug during manufacture. Furthermore, the formulation of the present invention can deliver large doses of API in small injection volumes as compared to small doses in large injection volumes for microspheres. Most importantly, the depot obtained with the formulation of the present invention protects sensitive biopharmaceuticals from in vivo degradation and enzymatic inactivation.

The formulation of the present invention is a patient-friendly delivery platform, when compared to other implantable or reservoir devices. The formulation of the present invention is injected subcutaneously and the resulting implant releases drug over a predetermined interval of time. Typically, the implant biodegrades at the same rate that the drug is released; therefore, the injection site essentially resolves in time for the next injection. In comparison, mechanical implants must be removed surgically and replaced or refilled after the drug reservoir is depleted.

When used to administer a biological agent to the eye, the flowable composition described herein employs substances in an effective and suitable amount, to diminish the occurrence and/or severity of irritation or toxicity to the eye and surrounding tissue. Such irritation or toxicity can be caused, e.g., by the presence of relatively large amounts of organic solvent, such as, e.g., acetone or N-methyl-2-pyrrolidone.

The present invention provides a flowable composition suitable for use as a controlled release implant, the composition includes: (a) a biodegradable, biocompatible thermoplastic polymer that is at least substantially insoluble in aqueous medium, water or body fluid; (b) a biological agent, a metabolite thereof, a biological agently acceptable salt thereof, or a prodrug thereof; and (c) a biocompatible organic liquid, at standard temperature and pressure, in which the thermoplastic polymer is soluble; wherein the composition is suitable for ocular delivery.

The present invention also provides a method of treating a disease or disorder in a mammal, the method includes administering to the ocular region of a mammal in need of such treatment an effective amount of the flowable composition of the present invention.

The present invention also provides a method for locally delivering a biological agent via the ocular region of a mammal, the method including contacting the ocular region of the mammal with the flowable composition of the present invention.

The present invention also provides a method for systemically delivering a biological agent via an ocular region of a mammal, the method including contacting the ocular region of the mammal with the flowable composition of the present invention.

The present invention also provides an implant that includes: (a) a biodegradable, biocompatible thermoplastic polymer that is at least substantially insoluble in aqueous medium, water or body fluid; (b) a biological agent, a metabolite thereof, a biological agently acceptable salt thereof, or a prodrug thereof; and (c) a biocompatible organic liquid at standard temperature and pressure, in which the thermoplastic polymer is soluble; wherein the implant is located in the ocular region of a mammal and the implant has a solid or gelatinous microporous matrix, the matrix being a core surrounded by a skin and wherein the implant is surrounded by body tissue.

The present invention also provides an implant that includes: (a) a biodegradable, biocompatible thermoplastic polymer that is at least substantially insoluble in aqueous medium, water or body fluid; and (b) a biological agent, a metabolite thereof, a biological agently acceptable salt thereof, or a prodrug thereof; wherein the implant is located in the ocular region of a mammal and the implant has a solid or gelatinous microporous matrix, the matrix being a core surrounded by a skin and wherein the implant is surrounded by body tissue.

The present invention also provides a method of forming an implant in situ within the ocular region of a living body, the method includes: (a) injecting a flowable composition within the ocular region of a patient, the flowable composition any one of the present invention; and (b) allowing the biocompatible organic liquid to dissipate to produce a solid biodegradable implant.

The present invention also provides a biological agent kit suitable for in situ formation of a biodegradable implant in an ocular region, the kit includes: (a) a first container comprising a flowable composition suitable for delivery into an ocular region, the composition comprising: (i) a biodegradable, biocompatible thermoplastic polymer that is at least substantially insoluble in aqueous medium, water or body fluid; and (ii) a biocompatible organic liquid at standard temperature and pressure, in which the thermoplastic polymer is soluble; (b) a second container comprising a biological agent, a metabolite thereof, a biological agently acceptable salt thereof, or a prodrug thereof.

### BRIEF DESCRIPTION OF THE DRAWINGS

Embodiments of the invention may be best understood by referring to the following description and accompanying drawings which illustrate such embodiments. The numbering scheme for the Figures included herein are such that the leading number for a given reference number in a Figure is associated with the number of the Figure. Reference numbers are the same for those elements that are the same across different Figures. For example, ocular regions and ocular surfaces, such as the lacrimal ducts (110) can be located in FIG. 1. However, reference numbers are the same for those elements that are the same across different Figures. In the drawings:

FIG. 1 illustrates ocular regions and ocular surfaces useful in the present invention.

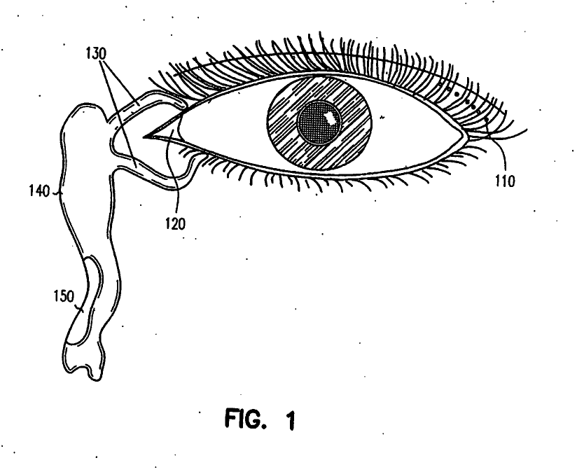

FIG. 2 illustrates ocular regions and ocular surfaces useful in the present invention.

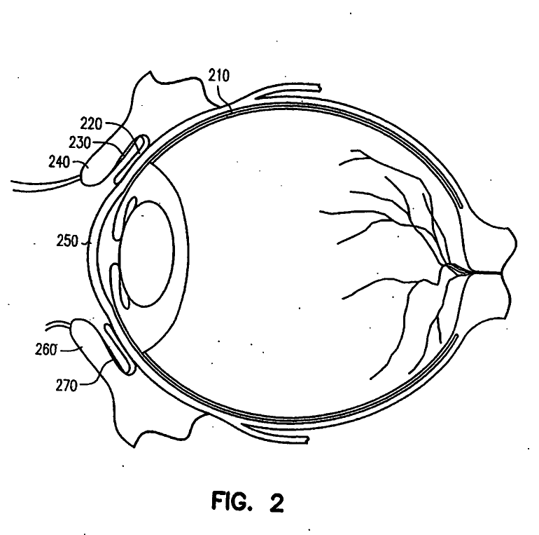

FIG. 3 illustrates ocular regions and ocular surfaces useful in the present invention.

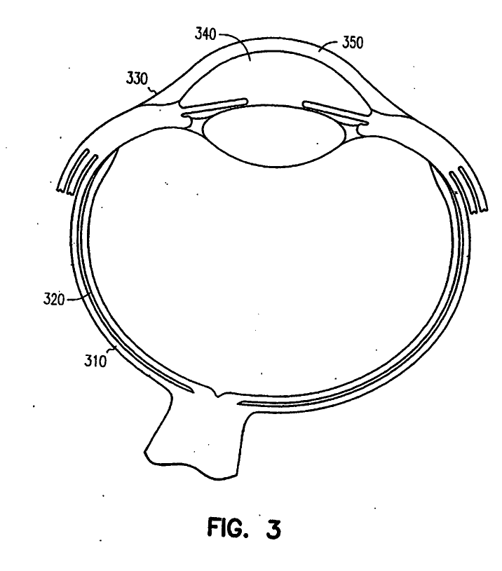

FIG. 4 illustrates mucosal regions and mucosal surfaces useful in the present invention.

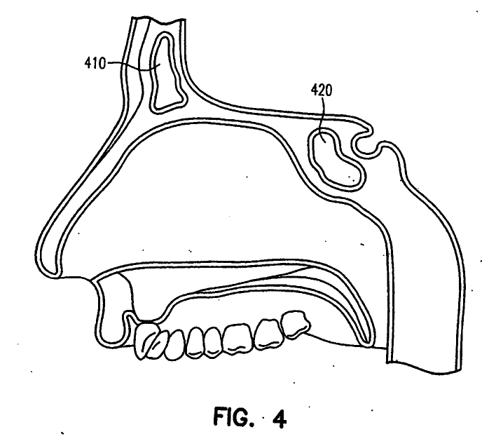

### DETAILED DESCRIPTION OF THE INVENTION

The present invention is directed to the ocular delivery of a flowable composition, suitable for use as a controlled release implant. The composition includes: (a) a biodegradable, biocompatible thermoplastic polymer that is at least substantially insoluble in aqueous medium, water or body fluid; (b) a biological agent, a metabolite thereof, a biological agently acceptable salt thereof, or a prodrug thereof; and (c) a biocompatible organic liquid, at standard temperature and pressure, in which the thermoplastic polymer is soluble. The thermoplastic polymer is at least substantially, preferably essentially completely soluble, in the organic solvent and is at least substantially, preferably completely insoluble in aqueous medium, body fluid and water. The organic solvent is at least slightly soluble in water, preferably moderately soluble in water, and especially preferably substantially soluble in water. The flowable composition is biological agently suitable for injection into a body wherein it will form a biological agently acceptable, solid matrix, which typically is a single body implant or drug delivery system. The implant will release the biological agent, metabolite thereof, biological agently acceptable salt thereof, or prodrug thereof, at a controlled rate. The rate of release may be altered to be faster or slower by inclusion of a rate-modifying agent.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, “ocular” or “ocular region” (550) refers to the eye, surrounding tissues, and to bodily fluids in the region of the eye. Specifically, the term includes the cornea (350) or (250), the sclera (310) or (210), the uvea (320), the conjunctiva (330) (e.g., bulbar conjunctiva (220), palpebral conjunctiva (230), and tarsal conjunctiva (270)), anterior chamber (340), lacrimal sac, lacrimal canals (130), lacrimal ducts (110), medial canthus (120), nasolacrimal duct (150), and the eyelids (e.g., upper eyelid (240) and lower eyelid (260)). Additionally, the term includes the inner surface of the eye (conjunctiva overlying the sclera (310) or (210)), and the inner surface of the eyelids (palpepral conjunctiva).

As used herein, “conjunctiva” refers to the mucous membrane lining the inner surfaces of the eyelids and anterior part of the sclera (310) or (210). The “palpebral conjunctiva” lines the inner surface of the eyelids and is thick, opaque, and highly vascular. The “bulbar conjunctiva” is loosely connected, thin, and transparent, covering the sclera (310) or (210) of the anterior third of the eye.

As used herein, “cornea” refers to the convex, transparent anterior part of the eye, comprising one sixth of the outermost tunic of the eye bulb. It allows light to pass through it to the lens. The cornea (350) or (250) is a fibrous structure with five layers: the anterior corneal epithelium, continuous with that of the conjunctiva; the anterior limiting layer (Bowman's membrane); the substantial propria; the posterior limiting layer (Descemet's membrane); and the endothelium of the anterior chamber (340) (keratoderma). It is dense, uniform in thickness, and nonvascular, and it projects like a dome beyond the sclera (310) or (210), which forms the other five sixths of the eye's outermost tunic. The degree of corneal curvature varies among different individuals and in the same person at different ages; the curvature is more pronounced in youth than in advanced age.

As used herein, “eye” refers to one of a pair of organs of sight, contained in a bony orbit at the front of the skull, embedded in orbital fat, and innervated by four cranial nerves: optic, oculomotor, trochlear, and abducens. Associated with the eye are certain accessory structures, such as the muscles, the fasciae, the eyebrow, the eyelids, the conjunctiva (330), and the lacrimal gland. The bulb of the eye is composed of segments of two spheres with nearly parallel axes that constitute the outside tunic and one of three fibrous layers enclosing two internal cavities separated by the crystalline lens. The smaller cavity anterior to the lens is divided by the iris into two chambers, both filled with aqueous humor. The posterior cavity is larger than the anterior cavity and contains the jellylike vitreous body that is divided by the hyaloid canal. The outside tunic of the bulb consists of the transparent cornea anteriorly, constituting one fifth of the tunic, and the opaque sclera posteriorly, constituting five sixths of the tunic. The intermediate vascular, pigmented tunic consists of the choroid, the ciliary body, and the iris. The internal tunic of nervous tissue is the retina. Light waves passing through the lens strike a layer of rods and cones in the retina, creating impulses that are transmitted by the optic nerve to the brain. The transverse and the anteroposterior diameters of the eye bulb are slightly greater than the vertical diameter; the bulb in women is usually smaller than the bulb in men. Eye movement is controlled by six muscles: the superior and inferior oblique muscles and the superior, inferior, medial, and lateral rectus muscles. Also called bulbus oculi, eyeball.

As used herein, “eyelid” refers to a movable fold of thin skin over the eye, with eyelashes and ciliary and meibomian glands along its margin. It consists of loose connective tissue containing a thin plate of fibrous tissue lined with mucous membrane (conjunctiva). The orbicularis oculi muscle and the oculomotor nerve control the opening and closing of the eyelid. The upper and lower eyelids are separated by the palpebral fissure. Also called palpebra.

As used herein, “canthus” refers to a corner of the eye, the angle at the medial and the lateral margins of the eyelids. The medial canthus (120) opens into a small space containing the opening to a lacrimal duct. Also called palpebral commissure.

As used herein, “mucus” refers to the viscous, slippery secretions of mucous membranes and glands, containing mucin, white blood cells, water, inorganic salts, and exfoliated cells.

As used herein, “nasal sinus” refers to any one of the numerous cavities in various bones of the skull, lined with ciliated mucous membrane continuous with that of the nasal cavity. The membrane is very sensitive; easily irritated, it may cause swelling that blocks the sinuses. The nasal sinus can include, e.g., the frontal sinus (410) or the spheroidal sinus (420).

As used herein, “lacrimal” refers to tears.

As used herein, “lacrimal duct” refers to one of a pair of channels through which tears pass from the lacrimal lake to the lacrimal sac of each eye. Also called lacrimal canaliculus.

As used herein, “palpebral conjunctiva” refers to the mucous membrane lining the inner surfaces of the eyelids and anterior part of the sclera (310) or (210). The “palpebral conjunctiva” lines the inner surface of the eyelids and is thick, opaque, and highly vascular. The “bulbar conjunctiva” is loosely connected, thin, and transparent, covering the sclera (310) or (210) of the anterior third of the eye.

As used herein, “retina” refers to a 10-layered, delicate nervous tissue membrane of the eye, continuous with the optic nerve, that receives images of external objects and transmits visual impulses through the optic nerve to the brain. The retina is soft and semitransparent and contains rhodopsin. It consists of the outer pigmented layer and the nine-layered retina proper. These nine layers, starting with the most internal, are the internal limiting membrane, the stratum opticum, the ganglion cell layer, the inner plexiform layer, the inner nuclear layer, the outer plexiform layer, the outer nuclear layer, the external limiting membrane, and the layer of rods and cones. The outer surface of the retina is in contact with the choroid; the inner surface with the vitreous body. The retina is thinner anteriorly, where it extends nearly as far as the ciliary body, and thicker posteriorly, except for a thin spot in the exact center of the posterior surface where focus is best. The photoreceptors end anteriorly in the jagged ora serrata at the ciliary body, but the membrane of the retina extends over the back of the ciliary processes and the iris. The retina becomes clouded and opaque if exposed to direct sunlight. See also Jacob's membrane, macula, optic disc.

As used herein, “retinochoroid” refers to an inflammation of the retina and choroid coat of the eye.

As used herein, “sclera” refers to the tough inelastic opaque membrane covering the posterior five sixths of the eyebulb. It maintains the size and form of the bulb and attaches to muscles that move the bulb. Posteriorly it is pierced by the optic nerve and, with the transparent cornea, makes up the outermost of three tunics covering the eyebulb.

As used herein, “sinus” refers to a cavity or channel, such as a cavity within a bone, a dilated channel for venous blood, or one permitting the escape of purulent material.

As used herein, “tarsal gland” refers to any one of numerous modified sebaceous glands on the inner surfaces of the eyelids. Acute localized bacterial infection of a tarsal gland may cause a sty or a chalazion.

As used herein, “tears” refers to a watery saline or alkaline fluid secreted by the lacrimal glands to moisten the conjunctiva.

As used herein, “uvea” refers to the fibrous tunic beneath the sclera (310) or (210) that includes the iris, the ciliary body, and the choroid of the eye.

As used herein, “vasculature” refers to the distribution of blood vessels in an organ or tissue.

Biological Agent

The biological agent(s) can be suitable for local delivery in the eye. Alternatively, the biological agent(s) can be suitable for systemic delivery via the eye.

The biological agent can include a single biological agent or a combination of biological agents. Examples of categories of biological agents that can be used, either alone or in combination include: adrenergic agent; adrenocortical steroid; adrenocortical suppressant; alcohol deterrent; aldosterone antagonist; amino acid; ammonia detoxicant; anabolic; analeptic; analgesic; androgen; anti-angiogenic; anesthesia, adjunt to; anesthetic; anorectic; antagonist; anterior pituitary suppressant; anthelmintic; antiacne agent; anti-adrenergic; anti-allergic; anti-amebic; anti-androgen; anti-anemic antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enxzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregaton inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; and zxanthine oxidase inhibitor.

Specific biological agents that are examples of the classes of biological agents disclosed above include, but are not limied to, Acebutolol; Acebutolol; Acyclovir; Albuterol; Alfentanil; Almotriptan; Alprazlam; Amiodarone; Amlexanox; Amphotericin B; Anecortave Acetate; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide and analogues thereof; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; rhuFab V2; Rofecoxib; Repaglinide; Risperidone; Rizatriptan; Ropinirole; Somatostatin and analogues thereof; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Visudyne; Zaleplon; Zanamivir; Zafirlukast; Zolmitriptan; and Zolpidem.

The amount of biological agent to be placed with the composition depends on the desired treatment dosage to be administered, although typically, the biological agent component will be present in about 0.001% to about 50% by weight of the flowable composition, and more specifically between about 0.005 and about 35% by weight of the flowable composition.

In one embodiment, the flowable composition of the present invention can include an antimigraine medication as the biological agent. The antimigraine medication can include, e.g., naratriptan, zolmitriptan, rizatriptan, frovatriptan, octreatide, sumatriptan or other “triptan” biological agent.

In another embodiment, the flowable composition of the present invention can include an antiangiogenic agent as the biological agent. The flowable composition can deliver to the retinochoroid the antiangiogenic agent, to effectively treat patients with diabetic retinopathy or macular degeneration.

In another embodiment, the flowable composition of the present invention can include an immunosuppressive as the biological agent, to effectively treat patients with uveitis.

In another embodiment, the flowable composition of the present invention can include an immunosuppresive or anti-inflammatory agent as the biological agent. The flowable composition can locally deliver to the tarsal conjunctiva (270) the immunosuppresive or the anti-inflammatory agent, to effectively treat vernal keratoconjunctivitis.

In another embodiment, the flowable composition of the present invention can include a would-healing medication as the biological agent. The flowable composition would effectively hold the biological agent in direct contact with a corneal wound.

In another embodiment, the flowable composition) of the present invention can include an antiviral agent, an antibiotic agent, an antifungal agent, or a combination thereof. The flowable composition would effectively treat infectious diseases (e.g., bacterial, viral, or fungal).

In another embodiment, the flowable composition of the present invention can include an antiviral agent. The flowable composition would deliver the antiviral agent to the cornea (350) or (250), thereby effectively treating patients afflicted with herpetic conjunctivitis or blepharitis.

As used herein, “treat” or “treating” refers to: (i) preventing a pathologic condition from occurring (e.g. prophylaxis) or symptoms related to the same; (ii) inhibiting the pathologic condition or arresting its development or symptoms related to the same; or (iii) relieving the pathologic condition or symptoms related to the same.

It is appreciated that those of skill in the art understand that the terms “soluble” and “insoluble” are relative terms. For example, a substance that has a solubility, in water, of about 1×10⁻⁴⁵ mg/L is relativelt insoluble in water. It none-the-less, has some (i.e., discrete and finite) solubility in water. It is because of this impresice terminology that Applicant employs the terms “solubility ranging from completely insoluble in any proportion to completely soluble in all proportions,” “at least partially water-soluble,” and “completely water-soluble” to describe the organic solvent/liquid.

It is also appreciated that those of skill in the art understand that the solubility of an organic solvent/liquid in boldily fluid can vary, e.g., on the specified bodily fluid and with the specified individual. Since Applicant is unaware of any universally accepted parameters to define an organic liquid/solvent in terms of its solubility in bodily fluids, Applicant has described the organic liquid/solvent in terms of its solubility in water. As such, when reference is made to the solubility of an organic liquid/solvent in water, it is appreciated that those of skill in the art understand that this is to give guidance and direction to an organic liquid/solvent with an equivalent solubility in bolidy fluids. This is so even though it is understood that not all organic liquids/solvents have the same solubility in water than they do in bodily fluids.

The term ester linkage refers to —OC(═O)— or —C(═O)O—; the term thioester linkage refers to —SC(═O)— or —C(═O)S—; the term amide linkage refers to —N(R)C(═O)— or —C(═O)N(R)—, the term phosphoric acid ester refers to —OP(═O)₂O—; the term sulphonic acid ester refers to —SO₂O— or —OSO₂—, wherein each R is a suitable organic radical, such as, for example, hydrogen, (C₁-C₂₀)alkyl, (C₃-C₆)cycloalkyl, (C₃-C₆)cycloalkyl(C₁-C₂₀)alkyl, aryl, heteroaryl, aryl(C₁-C₂₀)alkyl, or heteroaryl(C₁-C₂₀)alkyl.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C1-C6)alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York, John Wiley & sons, Inc., and references cited therein).

The term “peptide” describes a sequence of 2 to 35 amino acids (e.g. as defined hereinabove) or peptidyl residues. The sequence may be linear or cyclic. For example, a cyclic peptide can be prepared or may result from the formation of disulfide bridges between two cysteine residues in a sequence. Preferably a peptide comprises 3 to 20, or 5 to 15 amino acids. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right.

The term “saccharide” refers to any sugar or other carbohydrate, especially a simple sugar or carbohydrate. Saccharides are an essential structural component of living cells and source of energy for animals. The term includes simple sugars with small molecules as well as macromolecular substances. Saccharides are classified according to the number of monosaccharide groups they contain.

The term “polysaccharide” refers to a type of carbohydrate that contains sugar molecules that are linked together chemically, i.e., through a glycosidic linkage. The term refers to any of a class of carbohydrates whose are carbohydrates that are made up of chains of simple sugars. Polysaccharides are polymers composed of multiple units of monosaccharide (simple sugar).

The term “fatty acid” refers to a class of aliphatic monocarboxylic acids that form part of a lipid molecule and can be derived from fat by hydrolysis. The term refers to any of many long lipid-carboxylic acid chains found in fats, oils, and as a component of phospholipids and glycolipids in animal cell membranes.

The term “polyalcohol” refers to a hydrocarbon that includes one or more (e.g., 2, 3, 4, or 5) hydroxyl groups.

The term “carbohydrate” refers to an essential structural component of living cells and source of energy for animals; includes simple sugars with small molecules as well as macromolecular substances; are classified according to the number of monosaccharide groups they contain. The term refers to one of a group of compounds including the sugars, starches, and gums, which contain six (or some multiple of six) carbon atoms, united with a variable number of hydrogen and oxygen atoms, but with the two latter always in proportion as to form water; as dextrose, {C₆H₁₂O₆}. The term refers to a compound or molecule that is composed of carbon, oxygen and hydrogen in the ratio of 2H:1C:1O. Carbohydrates can be simple sugars such as sucrose and fructose or complex polysaccharide polymers such as chitin.

As used herein, “starch” refers to the complex polysaccharides present in plants, consisting of α-(1,4)-D-glucose repeating subunits and α-(1,6)-glucosidic linkages.

As used herein, “dextrin” refers to a polymer of glucose with intermediate chain length produced by partial degradation of starch by heat, acid, enzyme, or a combination thereof.

As used herein, “maltodextrin” or “glucose polymer” refers to non-sweet, nutritive saccharide polymer that consists of D-glucose units linked primarily by α,-1,4 bonds and that has a DE (dextrose equivalent) of less than 20. See, e.g., The United States Food and Drug Administration (21 C.F.R. paragraph 184.1444). Maltodextrins are partially hydrolyzed starch products. Starch hydrolysis products are commonly characterized by their degree of hydrolysis, expressed as dextrose equivalent (DE), which is the percentage of reducing sugar calculated as dextrose on dry-weight basis.

As used herein, “cyclodextrins” refers to a group of naturally occurring clathrates and products by the action of Bacillus macerans amylase on starch, e.g., α-, β-, and γ-cyclodextrins.

Flowable Composition

According to the present invention, a flowable composition is provided in which a biocompatible, biodegradable, thermoplastic polymer and a biological agent, a metabolite thereof, a biological agently acceptable salt thereof, or a prodrug thereof are dissolved or dispersed in a biocompatible organic solvent.

Upon contact with an aqueous medium, body fluid or water, the flowable composition solidifies to form an implant or implantable article. The implants and implantable articles that are formed from the flowable polymer compositions of the present invention are used for controlled drug release. The biological agent, metabolite thereof, biological agently acceptable salt thereof, or prodrug thereof is contained within the solidified polymer matrix when the flowable composition undergoes its transformation to an implant or implantable article. When the implant is present within a body, the metabolite thereof, biological agently acceptable salt thereof, or prodrug thereof is released in a sustained manner through diffusion through the polymer matrix, by direct dissolution at the implant surfaces and by degradation and erosion of the thermoplastic polymer.

Polymer

The biocompatible, biodegradable, thermoplastic polymers used according to the invention can be made from a variety of monomers which form polymer chains or monomeric units joined together by linking groups. These include polymers with polymer chains or backbones containing such linking groups as ester, amide, urethane, anhydride, carbonate, urea, esteramide, acetal, ketal, and orthocarbonate groups as well as any other organic functional group that can be hydrolyzed by enzymatic or hydrolytic reaction (i.e., is biodegradable by this hydrolytic action). These polymers are usually formed by reaction of starting monomers containing the reactant groups that will form these backbone linking groups. For example, alcohols and carboxylic acids will form ester linking groups. Isocyanates and amines or alcohols will respectively form urea or urethane linking groups.

According to the present invention, some fraction of one of these starting monomers will be at least trifunctional, and preferably multifunctional. This multifunctional character provides at least some branching of the resulting polymer chain. For example, when the polymer chosen contains ester linking groups along its polymer backbone, the starting monomers normally will be hydroxycarboxylic acids, cyclic dimmers of hydroxycarboxylic acids, cyclic trimers of hydroxycarboxylic acids, diols or dicarboxylic acids. The polymers of the present invention are obtained by inclusion of some fraction of a starting monomer that is at least multifunctional. In addition, the polymers of the present invention may incorporate more than one multifunctional unit per polymer molecule, and typically many multifunctional units depending on the stoichiometry of the polymerization reaction. Preferably, the polymers of the present invention incorporate at least one multifunctional unit per polymer molecule. A so-called star or branched polymer is formed when one multifunctional unit is incorporated in each polymer molecule. The biodegradable, biocompatible thermoplastic polymer of the present invention can be a linear polymer; or the biodegradable, biocompatible thermoplastic polymer of the present invention can be a branched polymer.

For example, for the ester linking group polymer described above, a dihydroxycarboxylic acid would be included with the first kind of starting monomer, or a triol and/or a tricarboxylic acid would be included with the second kind of starting monomer. Similarly, a triol, quatraol, pentaol, or hexaol such as sorbitol or glucose can be included with the first kind of starting monomer. The same rationale would apply to polyamides. A triamine and/or triacid would be included with starting monomers of a diamine and dicarboxylic acid. An amino dicarboxylic acid, diamino carboxylic acid or a triamine would be included with the second kind of starting monomer, amino acid. Any aliphatic, aromatic or arylalkyl starting monomer having the specified functional groups can be used according to the invention to make the branched thermoplastic polymers of the invention, provided that the polymers and their degradation products are biocompatible. The biocompatiblity specifications of such starting monomers are known in the art.

In particular, the monomers used to make the biocompatible thermoplastic branched polymers of the present invention will produce polymers or copolymers that are biocompatible and biodegradable. Examples of biocompatible, biodegradable polymers suitable for use as the biocompatible thermoplastic branched polymers of the present invention include polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), and copolymers, terpolymers, or combinations or mixtures of the above materials.

The polymer composition of the invention can also include polymer blends of the polymers of the present invention with other biocompatible polymers, so long as they do not interfere undesirably with the biodegradable characteristics of the composition. Blends of the polymer of the invention with such other polymers may offer even greater flexibility in designing the precise release profile desired for targeted drug delivery or the precise rate of biodegradability desired for structural implants such as for orthopedic applications.

The preferred biocompatible thermoplastic polymers or copolymers of the present invention are those which have a lower degree of crystallization and are more hydrophobic. These polymers and copolymers are more soluble in the biocompatible organic solvents than highly crystalline polymers such as polyglycolide or chitin, which have a high degree of hydrogen-bonding. Preferred materials with the desired solubility parameters are branched polylactides, polycaprolactones, and copolymers of these with glycolide in, which there are more amorphous regions to enhance solubility. Generally, the biocompatible, biodegradable thermoplastic polymer is substantially soluble in the organic solvents so that up to 50-60 wt % solids can be made. Preferably, the polymers used according to the invention are essentially completely soluble in the organic solvent so that mixtures up to 85-98 wt % solids can be made. The polymers also are at least substantially insoluble in water so that less than 0.1 g of polymer per mL of water will dissolve or disperse in water. Preferably, the polymers used according to the invention are essentially completely insoluble in water so that less than 0.001 g of polymer per mL of water will dissolve or disperse in water. At this preferred level, the flowable composition with a completely water miscible solvent will almost immediately transform to the solid polymer.

Solvent/Liquid

Liquids suitable for use in the flowable composition are biocompatible and are at least slightly soluble in aqueous medium, body fluid, or water. The organic liquid preferably is at least moderately soluble, more preferably very soluble, and most preferably soluble at all concentrations in aqueous medium, body fluid, or water. An organic liquid that is at least slightly soluble in aqueous or body fluid will allow water to permeate into the polymer solution over a period of time ranging from seconds to weeks and cause it to coagulate or solidify. The slightly soluble liquids will slowly diffuse from the flowable composition and typically will enable the transformation over a period of days to weeks, e.g. about a day to several weeks. The moderately soluble to very soluble organic liquids will diffuse from the flowable composition over a period of minutes to days so that the transformation will occur rapidly but with sufficient leisure to allow its manipulation as a pliable implant after its placement. The highly soluble organic liquids will diffuse from the flowable composition over a period of seconds to hours so that the transformation will occur almost immediately. The organic liquid preferably is a polar aprotic or polar protic organic solvent. Preferably, the organic solvent has a molecular weight in the range of about 30 to about 1000.

Although it is not meant as a limitation of the invention, it is believed that the transition of the flowable composition to a solid is the result of the dissipation of the organic liquid from the flowable composition into the surrounding aqueous medium or body fluid and the infusion of water from the surrounding aqueous medium or body fluid into the organic liquid within the flowable composition. It is believed that during this transition, the thermoplastic polymer and organic liquid within the flowable composition partition into regions rich and poor in polymer. The regions poor in polymer become infused with water and yield the porous nature of the resulting solid structure.

Examples of biocompatible organic liquids that may be used to form the flowable compositions of the present invention include aliphatic, aryl, and arylalkyl linear, cyclic and branched organic compounds that are liquid or at least flowable at ambient and physiological temperature and contain such functional groups as alcohols, ketones, ethers, amides, esters, carbonates, sulfoxides, sulfones, and any other functional group that is compatible with living tissue.

Preferred biocompatible organic liquids that are at least slightly soluble in aqueous or body fluid include N-methyl-2-pyrrolidone, 2-pyrrolidone; C₁ to C₁₅ alcohols, diols, triols and tetraols such as ethanol, glycerine, propylene glycol, butanol; C₃ to C₁₅ alkyl ketones such as acetone, diethyl ketone and methyl ethyl ketone; C₃ to C₁₅ esters such as methyl acetate, ethyl acetate, ethyl lactate; C₁ to C₁₅ amides such as dimethylformamide, dimethylacetamide and caprolactam; C₃ to C₂₀ ethers such as tetrahydrofuran, or solketal; tweens, triacetin, propylene carbonate, decylmethylsulfoxide, dimethyl sulfoxide, oleic acid, and 1-dodecylazacycloheptan-2-one. Other preferred organic liquids are benzyl alcohol, benzyl benzoate, dipropylene glycol, tributyrin, ethyl oleate, glycerin, glycofural, isopropyl myristate, isopropyl palmitate, oleic acid, polyethylene glycol, propylene carbonate, and triethyl citrate. The most preferred solvents are N-methyl-2-pyrrolidone, 2-pyrrolidone, dimethyl sulfoxide, triacetin, and propylene carbonate because of their solvating ability and their compatibility.

The solubility of the biodegradable thermoplastic polymers in the various organic liquids will differ depending upon their crystallinity, their hydrophilicity, hydrogen-bonding, and molecular weight. Lower molecular-weight polymers will normally dissolve more readily in the organic liquids than high-molecular-weight polymers. As a result, the concentration of a polymer dissolved in the various organic liquids will differ depending upon type of polymer and its molecular weight. Moreover, the higher molecular-weight polymers will tend to give higher solution viscosities than the low-molecular-weight materials.

Generally, the concentration of the polymer in the organic liquid according to the invention will range from about 0.01 g per ml of organic liquid to a saturated concentration. Typically, the saturated concentration will be in the range of 80 to 95 wt % solids or 4 to almost 5 gm per ml of organic liquid, assuming that the solvent weighs approximately 1 gm per ml.

For polymers that tend to coagulate slowly, a solvent mixture can be used to increase the coagulation rate. In essence, one liquid component of the solvent mixture is a good solvent for the polymer, and the other liquid component of the solvent mixture is a poorer solvent or a non-solvent. The two liquids are mixed at a ratio such that the polymer is still soluble but precipitates with the slightest increase in the amount of non-solvent, such as water in a physiological environment. By necessity, the solvent system must be miscible with both the polymer and water. An example of such a binary solvent system is the use of N-methylpyrrolidone and ethanol. The addition of ethanol to the NMP/polymer solution increases its coagulation rate.

The pliability of the composition can be substantially maintained throughout its life as an implant if a certain subgroup of the organic liquid of the composition is used. Such organic liquid also can act as a plasticizer for the thermoplastic polymer and at least in part may remain in the composition rather than dispersing into body fluid, especially when the organic liquid has low water solubility. Such an organic liquid having these low water solubility and plasticizing properties may be included in the composition in addition to the organic liquid that is highly water soluble. In the latter situation, the first organic liquid preferably will rapidly disperse into the body fluid.

Organic liquids of low water solubility, i.e. those forming aqueous solutions of no more than 5% by weight in water can also be used as the organic liquid of the implant composition. Such organic liquids can also act as plasticizers for the thermoplastic polymer. When the organic liquid has these properties, it is a member of a subgroup of organic solvents termed “plasticizer organic liquids” herein. The plasticizer organic liquid influences the pliablity and moldability of the implant composition such that it is rendered more comfortable to the patient when implanted. Moreover, the plasticizer organic liquid has an effect upon the rate of sustained release of the biologically active agent such that the rate can be increased or decreased according to the character of the plasticizer organic liquid incorporated into the implant composition. Although the organic liquid of low water solubility and plasticizing ability can be used alone as the organic liquid of the implant composition, it is preferable to use it in combination as follows. When a high water solubility organic liquid is chosen for primary use in the implant composition, the plasticizer effect can be achieved by use of a second organic liquid having a low water solubility and a plasticizing ability. In this instance, the second organic liquid is a member of the organic liquid subgroup and at least in part will remain in the implant composition for a sustained period. In general, the organic liquid acting as a plasticizer is believed to facilitate molecular movement within the solid thermoplastic matrix. The plasticizing capability enables polymer molecules of the matrix to move relative to each other so that pliability and easy moldability are provided. The plasticizing capability also enables easy movement of the bioactive agent so that in some situations, the rate of sustained release is either positively or negatively affected.

High Water Solubility Organic Liquids/Solvents

A highly water soluble organic liquid can be generally used in the implant composition and especially when pliability will not be an issue after implantation of the implant composition. Use of the highly water soluble organic liquid will produce an implant having the physical characteristics of and implant made through direct insertion of the flowable composition. Such implants and the precursor flowable compositions are described, for example in U.S. Pat. Nos. 4,938,763 and 5,278,201, the disclosures of which are incorporated herein by reference.

Useful, highly water soluble organic liquids include, for example, substituted heterocyclic compounds such as N-methyl-2-pyrrolidone (NMP) and 2-pyrrolidone; C₂ to C₁₀ alkanoic acids such as acetic acid and lactic acid, esters of hydroxy acids such as methyl lactate, ethyl lactate, alkyl citrate and the like; monoesters of polycarboxylic acids such as monomethyl succinate acid, monomethyl citric acid and the like; ether alcohols such as glycofurol, glycerol formal, isopropylidene glycol, 2,2-dimethyl-1,3-dioxolone-4-methanol; Solketal; dialkylamides such as dimethylformamide, dimethylacetamide; dimethylsulfoxide (DMSO) and dimethylsulfone; lactones such as epsilon, caprolactone and butyrolactone; cyclic alkyl amides such as caprolactam; and mixtures and combinations thereof. Preferred organic liquids include N-methyl-2-pyrrolidone, 2-pyrrolidone, dimethylsulfoxide, ethyl lactate, glycofurol, glycerol formal, and isopropylidene glycol.

Low Water Solubility Organic Liquids/Solvents

As described above, a low water solubility organic liquid may also be used in the implant composition. Preferably, a low water solubility liquid is used when it is desirable to have an implant that remains pliable and is extrudable. Also, the release rate of the biologically active agent can be affected under some circumstances through the use of an organic liquid of low water solubility. Typically such circumstances involve retention of the organic liquid within the implant product and its function as a plasticizer.

Examples of low water soluble organic liquids include esters of carbonic acid and aryl alcohols such as benzyl benzoate; C₄ to C₁₀ alkyl alcohols; C₁ to C₆ alkyl C₂ to C₆ alkanoates; esters of carbonic acid and alkyl alcohols such as propylene carbonate, ethylene carbonate and dimethyl carbonate, alkyl esters of mono-, di-, and tricarboxylic acids, such as 2-ethyoxyethyl acetate, ethyl acetate, methyl acetate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate; alkyl ketones such as methyl ethyl ketone; as well as other carbonyl, ether, carboxylic ester, amide and hydroxy containing liquid organic compounds having some solubility in water. Propylene carbonate, ethyl acetate, triethyl citrate, isopropyl myristate, and glyceryl triacetate are preferred because of biocompatitibility and biological agent acceptance.

Additionally, mixtures of the foregoing high and low water solubility organic liquids providing varying degrees of solubility for the matrix forming material can be used to alter the hardening rate of the implant composition. Examples include a combination of N-methylpyrrolidone and propylene carbonate, which provides a more hydrophobic solvent than N-methylpyrrolidone alone, and a combination of N-methyl pyrrolidone and polyethylene glycol, which provides a more hydrophilic solvent than N-methylpyrrolidone alone.

Prodrugs include hydroxyl and amino derivatives well-known to practitioners of the art, such as, for example, esters prepared by reaction of the parent hydroxyl compound with a suitable carboxylic acid, or amides prepared by reaction of the parent amino compound with a suitable carboxylic acid. Simple aliphatic or aromatic esters derived from hydroxyl groups pendent on the compounds employed in this invention are preferred prodrugs. In some cases it may be desirable to prepare double ester type prodrugs such as (acyloxy)alkyl esters or ((alkoxycarbonyl)oxy)alkyl esters. Specific suitable esters as prodrugs include methyl, ethyl, propyl, isopropyl, n-butyl, isobutyl, tert-butyl, and morpholinoethyl.

Hydrolysis in Drug and Prodrug Metabolism: Chemistry, Biochemistry, and Enzymology, by Bernard Testa and Joachim Mayer; Vch Verlagsgesellschaft Mbh (August 2003) provides a comprehensive review of metabolic reactions and enzymes involved in the hydrolysis of drugs and prodrugs. The text also describes the significance of biotransformation and discusses the physiological roles of hydrolytic enzymes, hydrolysis of amides, and the hydrolysis of lactams. Additional references useful in designing prodrugs employed in the present invention include, e.g., Biological Approaches to the Controlled Delivery of Drugs (Annals of the New York Academy of Sciences, Vol. 507), R. L. Juliano (editor) (February 1988); Design of Biobiological agent Properties through Prodrugs and Analogs, Edward B. Roche (editor), Amer Biological agent Assn (MacK) (June 1977); Prodrugs: Topical and Ocular Drug Delivery (Drugs and the Biological agent Sciences, Vol. 53), Kenneth B. Sloan (editor), Marcel Dekker (Mar. 17, 1992); Enzyme-Prodrug Strategies for Cancer Therapy, Roger G. Melton (editor), Richard J. Knox (editor), Plenum Press (February 1999); Design of Prodrugs, Hans Bundgaard (editor), Elsevier Science (February 1986); Textbook of Drug Design and Development, Povl Krogsgaard-Larsen, Hans Bundgaard (editor), Hardwood Academic Pub (May 1991); Conversion of Non-Toxic Prodrugs to Active, Anti-Neoplastic Drugs Selectively in Breast Cancer Metastases, Basse, Per H. (September 2000); and Marine lipids for produrgs, of compounds and other biological agent applications, M. Másson, T. Loftsson and G. G. Haraldsson, Die Pharmazie, 55 (3), 172-177 (2000);

Prodrugs employed in the present invention can include any suitable functional group that can be chemically or metabolically cleaved by solvolysis or under physiological conditions to provide the biologically acive compound. Suitable functional groups include, e.g., carboxylic esters, amides, and thioesters. Depending on the reactive functional group(s) of the biologically active compound, a corresponding functional group of a suitable linker precursor can be selected from the following table, to provide, e.g., an ester linkage, thioester linkage, or amide linkage in the prodrug.

| Functional Group on | | |
|---|---|---|
| Biologically Active | Functional Group on | Resulting Linkage in |
| Compound | Linker Precursor | Prodrug |
| —COOH | —OH | Ester |
| —COOH | —NHR | Amide |
| —COOH | —SH | Thioester |
| —OH | —COOH | Carboxylic Ester |
| —SH | —COOH | Thioester |
| —NHR | —COOH | Amide |
| —OH | —OP(═O)(OH)₂ | Phosphoric Acid Ester |
| —OH | —OP(═O)(OR)₂ | Phosphoric Acid Ester |
| —OH | —SO₂OH | Sulphonic Acid Ester |

Linker Precursor and Linking Group

A biologically acive compound can be linked to a suitable linker precursor to provide the prodrug. As shown above, the reactive functional groups present on the biologically active compound will typically influence the functional groups that need to be present on the linker precursor. The nature of the linker precursor is not critical, provided the prodrug employed in the present invention possesses acceptable mechanical properties and release kinetics for the selected therapeutic application. The linker precursor is typically a divalent organic radical having a molecular weight of from about 25 daltons to about 400 daltons. More preferably, the linker precursor has a molecular weight of from about 40 daltons to about 200 daltons.

The resulting linking group, present on the prodrug, may be biologically inactive, or may itself possess biological activity. The linking group can also include other functional groups (including hydroxy groups, mercapto groups, amine groups, carboxylic acids, as well as others) that can be used to modify the properties of the prodrug (e.g. for appending other molecules) to the prodrug, for changing the solubility of the prodrug, or for effecting the biodistribution of the prodrug).

Specifically, the linking group can be a divalent, branched or unbranched, saturated or unsaturated, hydrocarbon chain, having from 1 to 50 carbon atoms, wherein one or more (e.g. 1, 2, 3, or 4) of the carbon atoms is optionally replaced by (—O—) or (—NR—, wherein R can be hydrogen, alkyl, cycloalkyl alkyl, or aryl alkyl, and wherein the chain is optionally substituted on carbon with one or more (e.g. 1, 2, 3, or 4) substituents selected from the group of alkoxy, substituted alkoxy, cycloalkyl, substituted cycloalkyl, alkanoyl, alkanoyloxy, alkoxycarbonyl, alkylthio, substituted alkylthio, hydroxycarbonyl, azido, cyano, nitro, halo, hydroxy, oxo, carboxy, aryl, substituted aryl, aryloxy, substituted aryloxy, heteroaryl, substituted heteroaryl, heteroaryloxy, substituted heteroaryloxy, COOR, or NRR, wherein each R can independently be hydrogen, alkyl, cycloalkyl alkyl, or aryl alkyl.

The term “alkyl” refers to a monoradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, sec-butyl, n-hexyl, n-decyl, tetradecyl, and the like.

The alkyl can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “alkylene” refers to a diradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methylene, ethylene, n-propylene, iso-propylene, n-butylene, iso-butylene, sec-butylene, n-hexylene, n-decylene, tetradecylene, and the like.

The alkylene can optionally be substituted with one or more alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkoxy, halo, haloalkyl, hydroxy, hydroxyalkyl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkoxy, halo, haloalkyl, hydroxy, hydroxyalkyl, aryl, heteroaryl, heterocycle, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, □-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkoxy, halo, haloalkyl, hydroxy, hydroxyalkyl, aryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano

The term “heterocycle” refers to a saturated or partially unsaturated ring system, containing at least one heteroatom selected from the group oxygen, nitrogen, and sulfur, and optionally substituted with alkyl or C(═O)ORb, wherein Rb is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

Another class of heterocyclics is known as “crown compounds” which refers to a specific class of heterocyclic compounds having one or more repeating units of the formula [—(CH₂-)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “nitro” refers to —NO₂.

The term “trifluoromethyl” refers to —CF₃.

The term “trifluoromethoxy” refers to —OCF₃.

The term “cyano” refers to —CN.

The term “hydroxy” refers to —OH.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl and cyano. When a substituent is keto (i.e., ═O) or thioxo (i.e., ═S) group, then 2 hydrogens on the atom are replaced.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Specifically, the linking group can be a divalent peptide, amino acid, fatty acid, saccharide, polysaccharide, polyalcohol (e.g., PEG or PVA), starch, dextrin, maltodextrin, cyclodextrin, or carbohydrate. For example, the linking group can be a divalent peptide, amino acid, saccharide, polysaccharide, or polyalcohol.

In one specific embodiment of the present invention, the linking group itself can have biological activity. For example, the linking group can be a divalent bioactive peptide such as growth hormone releasing peptide (GHRP), luteinizing hormone-releasing hormone (LHRH), leuprolide acetate, somatostatin, bombesin, gastrin releasing peptide (GRP), calcitonin, bradykinin, galanin, melanocyte stimulating hormone (MSH), growth hormone releasing factor (GRF), amylin, tachykinins, secretin, parathyroid hormone (PTH), enkephalin, endothelin, calcitonin gene releasing peptide (CGRP), neuromedins, parathyroid hormone related protein (PTHrP), glucagon, neurotensin, adrenocorticotrophic hormone (ACTH), peptide YY (PYY), glucagon releasing peptide (GLP), vasoactive intestinal peptide (VIP), pituitary adenylate cyclase activating peptide (PACAP), motilin, substance P, neuropeptide Y (NPY), TSH, and analogs and fragments thereof. See, e.g., U.S. Pat. Nos. 6,221,958; 6,113,943; and 5,863,985.

In one specific embodiment of the present invention, the linking group can be lipophillic. In another specific embodiment of the present invention, the linking group can be hydrophilic.

A suitable class of prodrugs include compounds of formula (I):
D-X¹-L¹ (I)

wherein,
D is a mono radical of a biologically acive compound disclosed herein;
X¹ is a carboxylic ester linkage, an amide linkage, a thioester linkage, a phosphoric acid ester linkage, or a sulphonic acid ester linkage; and
L¹ is a linking group.

Another suitable class of prodrugs include compounds of formula (II):
[D-X¹-L¹ [symbol] ₙX² (II)

wherein,
each D is independently a mono- or di-radical of a biologically acive compound disclosed herein;
each X¹ is independently a carboxylic ester linkage, an amide linkage, a thioester linkage, a phosphoric acid ester linkage, or a sulphonic acid ester linkage;
each L¹ is independently a linking group;
X² is a carboxylic ester, an amide, a thioester, a phosphoric acid ester, or a sulphonic acid ester; and
n is about 1 to about 10,000.
   As shown above, a suitable class of prodrugs includes polymeric prodrugs of biologically active compounds disclosed herein. Depending on the reactive functional group(s) of the biologically active compound, one or more positions of the biologically active compound can be chosen to link the linker precursor to the biologically active compound, in a repeated fashion, thereby providing the polymeric prodrug.
   Dosages

The flowable composition is a liquid or a gel composition, suitable for injection into the ocular region of a patient. The amount of flowable composition administered will typically depend upon the desired properties of the controlled release implant. For example, the amount of flowable composition can influence the length of time in which the biological agent, a metabolite thereof, or a prodrug thereof is released from the controlled release implant. Additionally, the amount of flowable composition administered will typically depend upon the specific intended use (e.g., nature and stage/progression of the disease or disorder). Additionally, the amount of flowable composition administered will typically depend upon the number of controlled release implants formed (i.e., the number of flowable compositions administered). Specifically, up to about 200, up to about 100, up to about 50, up to about 25, or up to about 10 flowable compositions can be administered and up to about 200, up to about 100, up to about 50, up to about 25, or up to about 10 controlled release implants can be formed by the administration of those flowable compositions. Typically, as the number of flowable compositions administered increases, the amount of flowable composition administered will decrease. Likewise, as the number of flowable compositions administered decreases, the amount of flowable composition administered will typically increase.

Specifically, the composition can be used to formulate a one year delivery system of biological agent, metabolite thereof, biological agently acceptable salt thereof, or prodrug thereof. The composition can also be used to formulate a six month delivery system of biological agent, metabolite thereof, biological agently acceptable salt thereof, or prodrug thereof. The composition can also be used to formulate a three month delivery system of biological agent, metabolite thereof, biological agently acceptable salt thereof, or prodrug thereof. The composition can also be used to formulate a two month delivery system of biological agent, metabolite thereof, biological agently acceptable salt thereof, or prodrug thereof. The composition can also be used to formulate a one month delivery system of biological agent, metabolite thereof, biological agently acceptable salt thereof, or prodrug thereof.

Specifically, up to about 10 mL of the flowable composition can be administered. More specifically, up to about 5 mL, up to about 1 mL, or up to about 0.5 mL of the flowable composition can be administered.

When multiple controlled release implants are formed (i.e., multiple flowable compositions are administered) as described above, each flowable composition administered can include the same amount of biological agent, metabolite thereof, biological agently acceptable salt thereof, or prodrug thereof. Alternatively, when multiple controlled release implants are formed (i.e., multiple flowable compositions are administered) as described above, each flowable composition administered can include a different amount of biological agent, metabolite thereof, biological agently acceptable salt thereof, or prodrug thereof. Each of the flowable compositions can be administered in any suitable amount. Specifically, each of the flowable composition administered can be up to about 10 mL, up to about 5 mL, up to about 1 mL, up to about 0.5 mL, or up to about 0.1 mL.

The biological agent, metabolite thereof, biological agently acceptable salt thereof, or prodrug thereof can be present in any effective, suitable and appropriate amount. For example, the biological agent, metabolite thereof, biological agently acceptable salt thereof, or prodrug thereof can be present up to about 70 wt. % of the flowable composition, up to about 60 wt. % of the flowable composition, up to about 40 wt. % of the flowable composition, or up to about 20 wt. % of the flowable composition. Specifically, the biological agent, metabolite thereof, biological agently acceptable salt thereof, or prodrug thereof can be present up to about 10 wt. % of the flowable composition, up to about 5 wt. % of the flowable composition, up to about 1 wt. % of the flowable composition, or up to about 0.1 wt. % of the flowable composition.

As described above, when multiple controlled release implants are formed (i.e., multiple flowable compositions are administered), each flowable composition administered can include the same amount of biological agent, metabolite thereof, biological agently acceptable salt thereof, or prodrug thereof. Alternatively, when multiple controlled release implants are formed (i.e., multiple flowable compositions are administered), each flowable composition administered can include a different amount of biological agent, metabolite thereof, biological agently acceptable salt thereof, or prodrug thereof. In any event, each of the flowable composition administered can independently include the biological agent, metabolite thereof, biological agently acceptable salt thereof, or prodrug thereof in up to about 10 wt. % of the flowable composition, up to about 5 wt. % of the flowable composition, up to about 1 wt. % of the flowable composition, or up to about 0.1 wt. % of the flowable composition.

Specicfically, the flowable composition can have a volume of more than about 0.001 mL. Additionally, the flowable composition can have a volume of up to about 20.0 mL. Specifically, the flowable composition can have a volume of about 0.01 mL to about 10.0 mL, about 0.05 mL to about 1.5 mL, about 0.1 mL to about 1.0 mL, or about 0.2 mL to about 0.8 mL.

Specifically, the flowable composition can be formulated for administration less than about once per day. More specifically, the flowable composition can be formulated for administration less than about once per week, less than about once per month, more than about once per year, about once per week to about once per year, or about once per month to about once per year.

The flowable composition will effectively deliver the biological agent, metabolite thereof, biological agently acceptable salt thereof, or prodrug thereof to mammalian tissue at a suitable, effective, safe, and appropriate dosage. For example, the flowable composition can effectively deliver the biological agent, metabolite thereof, biological agently acceptable salt thereof, or prodrug thereof to mammalian tissue at a dosage of more than about 0.001 picogram/kilogram/day, more than about 0.01 picogram/kilogram/day, more than about 0.1 picogram/kilogram/day, or more than about 1 picogram/kilogram/day. Alternatively, the flowable composition can effectively deliver the biological agent, metabolite thereof, biological agently acceptable salt thereof, or prodrug thereof to mammalian tissue at a dosage of up to about 100 milligram/kilogram/day, up to about 50 milligram/kilogram/day, up to about 10 milligram/kilogram/day, or up to about 1 milligram/kilogram/day.

More specifically, the flowable composition can effectively deliver the biological agent, metabolite thereof, biological agently acceptable salt thereof, or prodrug thereof to mammalian tissue at a dosage of about 0.001 picogram/kilogram/day to about 100 milligram/kilogram/day; about 0.01 picogram/kilogram/day to about 50 milligram/kilogram/day; about 0.1 picogram/kilogram/day to about 10 milligram/kilogram/day; or about 1 picogram/kilogram/day to about 1 milligram/kilogram/day.

The biological agent, metabolite thereof, biological agently acceptable salt thereof, or prodrug thereof can be released from the controlled-release implant in any suitable manner. For example, the biological agent, metabolite thereof, biological agently acceptable salt thereof, or prodrug thereof can be released from the controlled-release implant with linear or first order kinetics. Alternatively, the biological agent, metabolite thereof, biological agently acceptable salt thereof, or prodrug thereof can be released from the controlled-release implant in a continuous zero order. Additionally, the biological agent, metabolite thereof, biological agently acceptable salt thereof, or prodrug thereof can be released from the controlled-release implant with little or no drug burst.

The delivery of the biological agent, metabolite thereof, biological agently acceptable salt thereof, or prodrug thereof to the mammalian tissue can be systemic and/or local. Specifically, the dosage can be deleivered locally. More specifically, the dosage can be delivered locally for a period of time of up to about 1 year. More specifically, the dosage can be delivered locally for a period of time of up to about 1 month, up to about 1 week, or more than about 1 day.

In addition to the biological agent, metabolite thereof, biological agently acceptable salt thereof, or prodrug thereof; the flowable composition and/or the implant of the present invention can optionally include at least one of an analgesic, anesthetic, anti-infective agent, anti-migraine agent, muscle relaxant, or sedative and hypnotic. The analgesic, anesthetic, anti-infective agent, gastrointestinal agent, anti-migraine agent, muscle relaxant, or sedative and hypnotic can be present in any suitable amount. See, e.g., Physician's Desk Reference, 55^{th} Edition (2001).

Suitable analgesics include, e.g., acetaminophen, phenylpropanolamine HCl, chlorpheniramine maleate, hydrocodone bitartrate, acetaminophen elixir, diphenhydramine HCl, pseudoephedrine HCl, dextromethorphan HBr, guaifenesin, doxylamine succinate, pamabron, clonidine hydrochloride, tramadol hydrochloride, carbamazepine, sodium hyaluronate, lidocaine, hylan, Arnica Montana, radix (mountain arnica), Calendula officinalis (marigold), Hamamelis (witch hazel), Millefolium (milfoil), Belladonna (deadly nightshade), Aconitum napellus (monkshood), Chamomilla (chamomile), Symphytum officinale (comfrey), Bellis perennis (daisy), Echinacea angustifolia (narrow-leafed cone flower), Hypericum perforatum (St. John's wort), Hepar sulphuris calcareum (calcium sulfide), buprenorphine hydrochloride, nalbuphine hydrochloride, pentazocine hydrochloride, acetylsalicylic acid, salicylic acid, naloxone hydrochloride, oral transmucosal fentanyl citrate, morphine sulfate, propoxyphene napsylate, propoxyphene hydrochloride, meperidine hydrochloride, hydromorphone hydrochloride, fentanyl transdermal system, levorphanol tartrate, promethazine HCl, oxymorphone hydrochloride, levomethadyl acetate hydrochloride, oxycodone HCl, oxycodone, codeine phosphate, isometheptene mucate, dichloralphenazone, butalbital, naproxen sodium, diclofenac sodium, misoprostol, diclofenac potassium, celecoxib, sulindac, oxaprozin, salsalate, diflunisal, naproxen, piroxicam, indomethacin, indomethacin sodium trihydrate, etodolac, meloxicam, ibuprofen, fenoprofen calcium, ketoprofen, mefenamic acid, nabumetone, tolmetin sodium, ketorolac tromethamine, choline magnesium trisalicylate, and rofecoxib.

Suitable anesthetics include: propofol, halothane, desflurane, midazolam HCl, epinephrine, levobupivacaine, etidocaine hydrochloride, ropivacaine HCl, chloroprocaine HCl, bupivacaine HCl, and lidocaine HCl.

Suitable anti-infective agents include, e.g., trimethoprim, sulfamethoxazole, clarithromycin, ganciclovir sodium, ganciclovir, daunorubicin citrate liposome, fluconazole, doxorubicin HCl liposome, foscarnet sodium, interferon alfa-2b, atovaquone, rifabutun, trimetrexate glucoronate, itraconazole, ciclofovir, azithromycin, delavirdine mesylate, efavirenz, nevirapine, lamivudine/zidovudine, zalcitabine, didanosine, stavudine, abacavir sulfate, amprenavir, indinavir sulfate, saquinavir, saquinavir mesylate, ritonavir, nelfinavir, chloroquine hydrochloride, metronidazole, metronidazole hydrochloride, iodoquinol, albendazole, praziquantel, thiabendazole, ivermectin, mebendazole sulfate, tobramycin sulfate, tobramycin, azetreonam, cefotetan disodium, cefotetan, loracarbef, cefoxitin, meropenem, imipenemand cilastatin, cefazolin, cefaclor, ceftibuten, ceftizoxime, cefoperazone, cefuroxumeaxetil, cefprozil, ceftazidime, cefotaxime sodium, cefadroxil monohydrate, cephalexin, cephalexin hydrochloride, cefuroxime, cefazolin, cefamandole nafate, cefapime hydrochloride, cefdinir, ceftriaxone sodium, cefixme, cefpodoxime proxetil, dirithromycin, erythromycin, erythromycin ethylsuccinate, erythromycin stearate, erythromycin, sulfisoxazole acetyl, troleandomycin, azithromycin, clindamycin, clindamycin hydrochloride, colistimethate sodium, quinupristin/dalfopristin, vancomycin hydrochloride, amoxicillin, amoxicillin/calvulanate/potassium, penicillin G benzathine, penicillin G procaine, penicillin G potassium, carbenicillin indanyl sodium, piperacillin sodium, ticarcillin disodium, clavulanate potassium, ampicillin sodium/sulbactam sodium, tazobactam sodium, tetracycline HCl, demeclocycline hydrochloride, doxycycline hyclate, minocycline HCl, doxycycline monohydrate, oxytetracycline HCl, hydrocortisone acetate, doxycycline calcium, amphotericin B lipid, flucytosine, griseofulvin, terbinafine hydrochloride, ketoconazole, chloroquine hydrochloride, chloroquine phosphate, pyrimethamine, mefloquine hydrochloride, atovaquone and proguanil hydrochloride, hydroxychloroquine sulfate, ethambutol hydrochloride, aminosalicylic acid, rifapentine, rifampin, isoniazid, pyrazinamide, ethionamide, interferon alfa-n3, famciclovir, rimantadine hydrochloride, foscamet sodium, interferon alfacon-1, ribavirin, zanamivir, amantadine hydrochloride, palivizumab, oseltamivir phosphate, valacyclovir hydrochloride, nelfinavir mesylate, stavudine, acyclovir, acyclovir sodium, rifabutin, trimetrexate glucuronate, linezolid, moxifloxacin, moxifloxacin hydrochloride, ciprofloxacin, ciprofloxacin hydrochloride, ofloxacin, levofloxacin, lomefloxacin hydrochloride, nalidixic acid, norfloxacin, enoxacin, gatifloxacin, trovafloxacin mesylate, alatrofloxacin, sparfloxacin, aztreonam, nitrofurantoin monohydrate/macrocrystals, cefepime hydrochloride, fosfomycin tromethamine, neomycin sulfate-polymyxin B sulfate, imipenem, cilastatin, methenamine, methenamine mandelate, phenyl salicylate, atropine sulfate, hyoscyamine sulfate, benzoic acid, oxytetracycline hydrochloride, sulfamethizole, phenazopyridine hydrochloride, and sodium acid phosphate, monohydrate.

Suitable homeopathic remedies include, e.g., cocculus indicus, conium maculatum, ambra grisea, and petroleum.

Suitable anti-migraine agents include, e.g., timolol maleate, propranolol hydrochloride, dihydroergotamine mesylate, ergotamine tartrate, caffeine, divalproex sodium, acetaminophen, acetylsalicylic acid, salicylic acid, naratriptan hydrochloride, sumatriptan succinate, sumatriptan, rizatriptan benzoate, and zolmitriptan.

Suitable muscle relaxants include, e.g., succinylcholine chloride, vecuronium bromide, rapacuronium bromide, rocuronium bromide, dantrolene sodium, cyclobanzaprine HCl, orphenadrine citrate, chlorzoxazone, methocarbamol, acetylsalicylic acid, salicylic acid, metaxalone, carisoprodol, codeine phosphate, diazepam, and tizanidine hydrochloride.

Suitable sedatives and hypnotics include, e.g., mephobarbital, pentobarbital sodium, lorazepam, triazolam, estazolam, diazepam, midazolam HCl, zolpidem tartrate, melatonin, vitamin B12, folic acid, propofol, meperidine HCl, promethazine HCl, diphenhydramine HCl, zaleplon, and doxylamine succinate.

Diseases or Disorders of the Eye

The flowable composition described herein can be locally administered, via the ocular region, to treat one or more eye diseases or disorders. Suitable eye diseases or disorders include, e.g., Acute Zonal Occult Outer Retinopathy, Adie Syndrome, Age Related Macular Degeneration (AMD), Albinism, Amaurosis Fugax, Amblyopia, Aniridia, Anisocoria, Anophthalmos, Aphakia, Artery Occlusion, Astigmatism, Basal Cell Carcinoma, Blepharitis, Branch Retinal Artery Occlusion, Branch Retinal Vein Occlusion, Blepharoptosis, Blepharospasm, Blindness, Cataract, Cellophane Retinopathy, Central Retinal Vein Occlusion, Central Serous Chorioretinopathy, Chalazion, Chemical Burn, Choroidal Neovascular Membrane, Choroidal Nevus, Cogan's Dystrophy, Color Blindness, Computer Vision Syndrome, Conjunctivitis, Corneal Dystrophy, Corneal Edema, Corneal Ulcer, Cystoid Macular Edema, Cytomegalovirus, Chorioretinitis, Choroideremia, Coloboma, Dacryocystitis, Diabetic Retinopathy, Droopy Eyelids, Dry Eyes, Diplopia, Distichiasis, Duane Retraction Syndrome, Ectropion, Entropion, Epi-retinal membrane, Episcleritis, Esotropia, Exfoliation Syndrome, Exotropia, Eye Hemorrhage, Eye Neoplasms, Farsightedness, Flashes & Floaters, Foreign Body, Fuchs' Dystrophy, Giant Cell Arteritis, Glaucoma, General Fibrosis Syndrome, Gyrate Atrophy, Headaches, Herpes Simplex, Herpes Zoster, High Pressure in the Eye, Histoplasmosis (Ocular), Hyperopia, Hyphema, Hemianopsia, Hermanski-Pudlak Syndrome, Hordeolum, Homer Syndrome, Inward Turned Eyelid, Iris Neovascularization, Iris Nevus, Iritis, Keratoconus, Kearns-Sayer Syndrome, Keratitis, Lacrimal Apparatus Diseases, Lacrimal Duct Obstruction, Macular Degeneration, Macular Edema, Macular Hole, Macular Pucker, Marginal Blepharitis, Myopia, Microphthalmos, Myopia, Nystagmus, Nearsightedness, Neovascularization of the Cornea, Neovascularization of the Optic Nerve Head, Nevus (Choroidal), Nevus (Iris), Ocular Histoplasmosis, Ocular Rosacea, Optic Neuritis, Outward Turned Eyelid, Ophthalmoplegia, Optic Atrophies, Optic Neuropathy, Orbital Cellulitis, Pinguecula, Pink Eye, Posterior Capsular Opacification, Presbyopia, Pterygium, Ptosis, Papilledema, Peter's Anomaly, Recurrent Corneal Erosion, Red Eyes, Retinal Tear, Retinal Detachment, Retinitis Pigmentosa, Retinopathy of Prematurity, Retrolental Fibroplasia (ROP), Rubeosis, Retinal Vein Occlusion, Retinoschisis, Scleritis, Strabismus, Stye, Subconjunctival Hemorrhage, Scotoma, Strabismus, Temporal Arteritis, Thygeson's Superficial Punctate Keratitis, Trachoma Uveitis, Vein Occlusion, and Vitreous Detachment.

When the flowable compositions described herein are locally administered, via the ocular region, to treat one or more eye diseases or disorders, the flowable compositions will typically include one or more biological agents known to treat such eye diseases or disorders. Such suitable biological agents include, e.g., acetylcholine blocking agents (e.g., botox purified neurotoxin complex), adrenergic agonists (e.g., alphagan p, naphcon-a), antibiotics (e.g., polytrim, tobradex), antiglaucoma agents (e.g., betimol, betoptic s, cosopt, timoptic in ocudose, timoptic, timoptic-xe, azopt, cosopt, daranide, trusopt, lumigan, travatan, xalatan, alphagan P, naphcon-A, rev-eyes), antihistamine & mast cell stabilizer combinations (e.g., elesat, patanol, zaditor), antihistamines & combinations (e.g., naphcon-A, optivar), anti-infectives (e.g., polytrim, tobradex, ciloxan, quixin, vigamox, zymar, blephamide), anti-inflammatory agents (e.g., acular, acular ls, acular pf, voltaren, blephamide, tobradex), artificial tears/lubricants & combinations (e.g., bion tears, lacrisert, restasis, tears naturale forte, tears naturale free), beta adrenergic blocking agents (e.g., betimol, betoptic s, cosopt, timoptic in ocudose, timoptic, timoptic-xe), beta adrenergic blocking agent & carbonic anhydrase inhibitor combinations (e.g., cosopt), carbonic anhydrase inhibitors (e.g., azopt, cosopt, daranide, trusopt), decongestants (e.g., alphagan p, naphcon-a), agents for glaucoma (e.g., betimol, betoptic s, cosopt, timoptic in ocudose, timoptic, timoptic-xe, azopt, cosopt, daranide, trusopt, lumigan, travatan, xalatan, alphagan p, naphcon-a, rev-eyes), lubricants (e.g., bion tears, lacrisert, restasis, tears naturale forte, tears naturale free), mast cell stabilizers (e.g., alamast), photodynamic therapy agents (e.g., visudyne), prostaglandins (e.g., lumigan, travatan, xalatan), sympathomimetics & combinations (e.g., alphagan p, naphcon-a), vasoconstrictors (e.g., alphagan p, naphcon-a), vitamins & combinations (e.g., catasod-ocuxtra/optigold/macutein, visutein), antibiotics & combinations (e.g., polytrim, tobradex), quinolones (e.g., ciloxan, quixin, vigamox, zymar), sulfonamides & combinations (e.g., blephamide), miotics (e.g., rev-eyes), nonsteroidal anti-inflammatory drugs (e.g., acular, acular Is, acular pf, voltaren), and steroidal anti-inflammatory agents & combinations (e.g., blephamide, tobradex).

The flowable composition and/or the implant of the present invention can further include at least one of: a release rate modification agent for controlling the rate of release of the biological agent in vivo from an implant matrix; a pore-forming agent; a biodegradable, crystallization-controlling agent; a plasticizer; a leaching agent; a penetration enhancer; an absorption altering agent; an opacification agent; and a colorant.

Release Rate Modification Agent

Rate modifying agents, plasticizers and leachable agents can be included to manage the rate of release of bioactive agent and the pliability of the matrix. Known plasticizers as well as organic compounds that are suitable for secondary pseudobonding in polymer systems are acceptable as pliability modifiers and leaching agents. Generally these agents are esters of mono, di and tricarboxylic acids, diols and polyols, polyethers, non-ionic surfactants, fatty acids, fatty acid esters, oils such as vegetable oils, and the like. The concentrations of such agents within the solid matrix can range in amount up to 60 wt % relative to the total weight of the matrix, preferably up to 30 wt % and more preferably up to 15 wt %. Generally, these leaching agents, plasticizers and pliability modifiers and their application are described in U.S. Pat. Nos. 5,702,716 and 5,447,725, the disclosures of which are incorporated herein by reference with the proviso that the polymers to be used are the biocompatible, biodegradable, thermoplastic polymers of the present invention.

A release rate modification agent may also be included in the flowable composition for controlling the rate of breakdown of the implant matrix and/or the rate of release of a bioactive agent in vivo from the implant matrix. The rate modifying agent can increase or retard the rate of release depending upon the nature of the rate modifying agent incorporated into the solid matrix according to the invention. Examples of suitable substances for inclusion as a release rate modification agent include dimethyl citrate, triethyl citrate, ethyl-heptanoate, glycerin, hexanediol, and the like.

The polymer solution may include a release rate modification agent to provide controlled, sustained release of a bioactive agent from the implant matrix. Although not intended to be a limitation to the present disclosure, it is believed the release rate modification agent alters the release rate of a bioactive agent from the implant matrix by changing the hydrophobicity of the polymer implant.

The use of a release rate modification agent may either decrease or increase the release of the bioactive agent in the range of multiple orders of magnitude (e.g., 1 to 10 to 100), preferably up to a ten-fold change, as compared to the release of a bioactive agent from a solid matrix without the release rate modification agent. Release rate modification agents which are hydrophilic, such as polyethylene glycol, may increase the release of the bioactive agent. By an appropriate choice of the polymer molecular weight in combination with an effective amount of the release rate modification agent, the release rate and extent of release of a bioactive agent from the implant matrix may be varied, for example, from relatively fast to relatively slow.

Useful release rate modification agents include, for example, organic substances which are water-soluble, water-miscible, or water insoluble (i.e., water immiscible), with water-insoluble substances preferred.

The release rate modification agent is preferably an organic compound which will substitute as the complementary molecule for secondary valence bonding between polymer molecules, and increases the flexibility and ability of the polymer molecules to slide past each other. Such an organic compound preferably includes a hydrophobic and a hydrophilic region so as to effect secondary valence bonding. It is preferred that a release rate modification agent is compatible with the combination of polymers and solvent used to formulate polymer solution. It is further preferred that the release rate modification agent is a biological agently-acceptable substance.

Useful release rate modification agents include, for example, fatty acids, triglycerides, other like hydrophobic compounds, organic solvents, plasticizing compounds and hydrophilic compounds. Suitable release rate modification agents include, for example, esters of mono-, di-, and tricarboxylic acids, such as 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, and the like; polyhydroxy alcohols, such as propylene glycol, polyethylene glycol, glycerin, sorbitol, and the like; fatty acids; triesters of glycerol, such as triglycerides, epoxidized soybean oil, and other epoxidized vegetable oils; vegetable oils obtained from seeds, flowers, fruits, leaves, or stem of a plant or tree, such as sesame oil, soybean oil, cotton seed oil, almond oil, sunflower oil, and peanut oil; sterols, such as cholesterol; alcohols, such as C₆-C₁₂ alkanols, 2-ethoxyethanol, and the like. The release rate modification agent may be used singly or in combination with other such agents. Suitable combinations of release rate modification agents include, for example, glycerin/propylene glycol, sorbitol/glycerine, ethylene oxide/propylene oxide, butylene glycol/adipic acid, and the like. Preferred release rate modification agents include dimethyl citrate, triethyl citrate, ethyl heptanoate, glycerin, and hexanediol.

The amount of the release rate modification agent included in the polymer solution will vary according to the desired rate of release of the bioactive agent from the implant matrix. Preferably, the polymer solution contains about 0.5-15%, preferably about 5-10%, of a release rate modification agent.

Pore Forming Agent/Additive

The flowable composition of the present invention can be used for implantation, injection, or otherwise placed totally or partially within the body. One of the biologically active substances of the composition and the polymer of the invention may form a homogeneous matrix, or one of the biologically active substances may be encapsulated in some way within the polymer. For example, the one of the biologically active substances may be first encapsulated in a microsphere and then combined with the polymer in such a way that at least a portion of the microsphere structure is maintained. Alternatively, one of the biologically active substances may be sufficiently immiscible in the polymer of the invention that it is dispersed as small droplets, rather than being dissolved, in the polymer. Either form is acceptable, but it is preferred that, regardless of the homogeneity of the composition, the release rate of that biologically active substance in vivo remain controlled, at least partially as a function of hydrolysis of the ester bond of the polymer upon biodegradation.

Additives can be used to advantage in further controlling the pore size in the solid matrix, which influences the structure of the matrix and the release rate of a bioactive agent or the diffusion rate of body fluids. For example, if the flowable composition is too impervious to aqueous medium, water or tissue ingrowth, a pore-forming agent can be added to generate additional pores in the matrix. Any biocompatible water-soluble material can be used as the pore-forming additive. These additives can be either soluble in the flowable composition or simply dispersed within it. They are capable of dissolving, diffusing or dispersing out of both the coagulating polymer matrix whereupon pores and microporous channels are generated. The amount of pore-forming additive (and size of dispersed particles of such pore-forming agent, if appropriate) within the flowable composition will directly affect the size and number of the pores in the polymer matrix.

Pore-forming additives include any biological agently acceptable organic or inorganic substance that is substantially miscible in water and body fluids and will dissipate from the forming and formed matrix into aqueous medium or body fluids or water-immiscible substances that rapidly degrade to water soluble substances. It is further preferred that the pore-forming additive is miscible or dispersible in the organic solvent to form a uniform mixture. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores can be varied over a wide range by changing the molecular weight and percentage of pore-forming additive incorporated into the flowable composition.

As indicated, upon contact with body fluid, the solvent and optional pore-forming additive dissipate into surrounding tissue fluids. This causes the formation of microporous channels within the coagulating polymer matrix. Optionally, the pore-forming additive may dissipate from the matrix into the surrounding tissue fluids at a rate slower than that of the solvent, or be released from the matrix over time by biodegradation or bioerosion of the matrix. Preferably, the pore-forming additive dissipates from the coagulating implant matrix within a short time following implantation such that a matrix is formed with a porosity and pore structure effective to perform the particular purpose of the implant, as for example, a barrier system for a tissue regeneration site, a matrix for timed-release of a drug or medicament, and the like.

Porosity of the solid polymer matrix may be varied by the concentration of water-soluble or water-miscible ingredients, such as the solvent and/or pore-forming agent, in the polymer composition. For example, a high concentration of water-soluble substances in the flowable composition may produce a polymer matrix having a high degree of porosity. The concentration of the pore-forming agent relative to polymer in the composition may be varied to achieve different degrees of pore-formation, or porosity, in the matrix. Generally, the polymer composition will include about 0.01-1 gram of pore-forming agent per gram polymer.

The size or diameter of the pores formed in the matrix of the implant may be modified according to the size and/or distribution of the pore-forming agent within the polymer matrix. For example, pore-forming agents that are relatively insoluble in the polymer mixture may be selectively included in the polymer composition according to particle size in order to generate pores having a diameter that corresponds to the size of the pore-forming agent. Pore-forming agents that are soluble in the polymer mixture may be used to vary the pore size and porosity of the implant matrix by the pattern of distribution and/or aggregation of the pore-forming agent within the polymer mixture and coagulating and solid polymer matrix.

Pore diameter and distribution within the polymer matrix of the implant may be measured, as for example, according to scanning electron microscopy methods by examination of cross-sections of the polymer matrix. Porosity of the polymer matrix may be measured according to suitable methods known in the art, as for example, mercury intrusion porosimetry, specific gravity or density comparisons, calculation from scanning electron microscopy photographs, and the like. Additionally, porosity may be calculated according to the proportion or percent of water-soluble material included in the polymer composition. For example, a polymer composition which contains about 30% polymer and about 70% solvent and/or other water-soluble components will generate an implant having a polymer matrix of about 70% porosity.

The biologically active substance of the composition and the polymer of the invention may form a homogeneous matrix, or the biologically active substance may be encapsulated in some way within the polymer. For example, the biologically active substance may be first encapsulated in a microsphere and then combined with the polymer in such a way that at least a portion of the microsphere structure is maintained. Alternatively, the biologically active substance may be sufficiently immiscible in the polymer of the invention that it is dispersed as small droplets, rather than being dissolved, in the polymer. Either form is acceptable, but it is preferred that, regardless of the homogeneity of the composition, the release rate of the biologically active substance in vivo remain controlled, at least partially as a function of hydrolysis of the ester bond of the polymer upon biodegradation.

The article of the invention is designed for implantation or injection into the body of a mammal. It is particularly important that such an article result in minimal tissue irritation when implanted or injected into vasculated tissue. As a structural medical device, the polymer compositions of the invention provide a physical form having specific chemical, physical, and mechanical properties sufficient for the application and a composition that degrades in vivo into non-toxic residues.

The implant formed within the injectable polymer solution will slowly biodegrade within the body and allow natural tissue to grow and replace the impact as it disappears. The implant formed from the injectable system will release the drug contained within its matrix at a controlled rate until the drug is depleted. With certain drugs, the polymer will degrade after the drug has been completely released. With other drugs such as peptides or proteins, the drug will be completely released only after the polymer has degraded to a point where the non-diffusing drug has been exposed to the body fluids.

Biodegradable, Crystallization-Controlling Agent

A crystallization-controlling agent may optionally be combined with the polymer to effect homogeneity of the polymer mass, that is, a substantially uniform distribution of crystalline sections of the polymer to achieve a homogeneous mass having the desired physical characteristics of moldability, cohesion, and stability for effective use with bone and other tissues. The crystallization-controlling agent may be in the form of a dispersed solid particle in the composition, for example, an inorganic salt such as calcium carbonate or calcium phosphate, a polymer such as poly(vinyl alcohol), starch or dextran, and other like substance. Other useful crystallization-controlling agent are those substances that are either melted with the polymer during the compounding process, or soluble in the molten polymer. Examples of those substances include low molecular weight organic compounds such as glycerol palmitate or ethyl lactate, polymers such as poly(ethylene glycol) or poly(lactide-co-caprolactone), and other like substances. Compositions formulated with a crystallization-controlling agent include about 40-95 wt-% of the polymer, preferably about 60-90 wt-%, and about 5-60 wt-% of the crystallization-controlling agent, preferably about 10-40 wt-%.

Crystallization-controlling agents suitable for use in the present compositions may be divided into two major classes, those that persist in the form of a solid particulate in the molten composition, and those that melt or dissolve in the molten polymer composition.

Crystallization-controlling agents that will persist as solid particles, or fillers, in the composition include inorganic or organic salts, and polymers. Suitable inorganic salts include, for example, calcium carbonate, hydroxy apatite, calcium phosphate, calcium apatite, calcium sulfate, calcium bicarbonate, calcium chloride, sodium carbonate, sodium bicarbonate, sodium chloride, and other like salts. Suitable organic salts include for example, calcium stearate, calcium palmitate, sodium stearate, other metallic salts of C₁₀-C₅₀ fatty acid derivatives, and other like salts. Polymers suitable for use in the composition that persist as dispersed particles or fillers in the composition include, for example, polysaccharides, cellulose derivatives and poly(vinyl alcohol). Examples of suitable polysaccharides include, for example, dextran, maltodextrin, starches derived from corn, wheat, rice and the like, and starch derivatives such as sodium starch glycolate. Examples of suitable cellulose derivatives include for example, sodium carboxymethyl cellulose, crosslinked sodium carboxymethyl cellulose, carboxylmethyl cellulose, hydroxyethyl cellulose, and the like. Suitable poly(vinyl alcohol)s have a molecular weight of about 5,000 to 20,000, preferably about 10,000-15,000, with a percent hydrolysis of about 80-100%.

Crystallization-controlling agents which either melt with or dissolve into the molten polymer during compounding may also be used in the polymer compositions of the invention. These compositions may or may not undergo some degree of phase separation during cooling. Crystallization-controlling agents of this type include low molecular weight organic compounds and polymers. Suitable low molecular weight compounds include, for example, glycerol, palmitate, glycerol stearate and other like glycerol derivatives, triethyl citrate and other like citric acid derivatives, ethyl lactate and other like esters, and the like.

The crystallization-controlling agent is included in the composition in an amount effective to soften the polymer to a moldable and/or smearable consistency. Preferably, the crystallization-controlling agent is a non-solvent, solid substance. A crystallization-controlling agent may be included in the composition alone or in combination with another crystallization-controlling agent. An example of a preferred combination of such agents is poly(lactide-co-caprolactone) and calcium stearate.

Penetration Enhancer

The composition may further comprise a penetration enhancer effective to improve the penetration of the biological agent into and through bodily tissue, with respect to a composition lacking the penetration enhancer. The penetration enhancer may generally be any penetration enhancer, preferably is oleic acid, oleyl alcohol, ethoxydiglycol, laurocapram, alkanecarboxylic acids, dimethylsulfoxide, polar lipids, or N-methyl-2-pyrrolidone, and more preferably is oleic acid or oleyl alcohol. The penetration enhancer can be present in the flowable composition in any suitable and appropriate amount (e.g., between about 1 wt. % and about 10 wt. %)

Absorption Altering Agent

Any suitable and appropriate absorption altering agent can be employed in the present invention. For example, the absorption altering agent can be selected from the group of propylene glycol, glycerol, urea, diethyl sebecate sodium, lauryl sulfate, sodium lauryl sulfate, sorbitan ethoxylates, oleic acid, pyrrolidone carboxylate esters, N-methylpyrrolidone, N,N-diethyl-m-tolumide, dimethyl sulfoxide, alkyl methyl sulfoxides, and combinations thereof.

Opacification Agent

Any suitable and appropriate opacification agent can be employed in the present invention. For example, the opacification agent can be selected from the group of barium, iodine, calcium, and any combination thereof.

Colorant

Colorants can also be added to the liquid composition in an amount effective to allow monitoring of the biodegradability or bioerodibility of the microporous film over time. Suitable and appropriate colorants will be nontoxic, non-irritating and non-reactive with the solvent in the liquid composition. Colorants which have been approved by the FDA for use in cosmetics, foods and drugs include: D & C Yellow No. 7; D & C Red No. 17; D & C Red No. 7, 9, and 34; FD & C Red No. 4; Orange D & C No. 4; FD & C Blue 2; FD & C Green No. 3, and the like.

Moldable Implant Precursor

The flowable composition can be formed into a moldable implant precursor by its contact with an aqueous medium such as water or saline, or contact with a body fluid such as blood serum, lymph, and the like pursuant to the techniques disclosed in U.S. Pat. No. 5,487,897, the disclosure of which is incorporated herein by reference with the specification that the thermoplastic polymer of the '897 patent is a biocompatible, biodegradable, thermoplastic polymer as described herein.

Briefly, the technique disclosed by the '897 patent converts the flowable composition with or without bioactive agent into a two-part structure comprising an outer sac with a flowable content. The technique applies a limited amount of aqueous medium and the like to a quantity of the biological agent system so that only the outer surface of the system is converted to solid, thus forming the sac with a flowable content inside. The flowable content of the implant precursor may range in consistency from watery to viscous. The outer sac may range in consistency from gelatinous to an impressionable, moldable and waxen-like. The resulting device, or implant precursor, may then be applied to an implant site. Upon implantation, the solvent from the implant precursor diffuses into the surrounding tissue fluids to form an implant having a solid polymer matrix. Preferably, the implant precursor solidifies in situ to a solid matrix within about 0.5-4 hours after implantation, preferably within about 1-3 hours, preferably within about 2 hours. Thus, when placed into an implant site in a body, the implant precursor eventually coagulates to a solid, microporous matrix structure.

Porous Structure

The porous structure of the solid matrices, e.g., in situ formed implants, implants, implantable articles, biodegradable articles and devices of the invention, is influenced by nature of the organic solvent and thermoplastic polymer, by their solubility in water, aqueous medium or body fluid (which may differ for each medium) and by the presence of an additional substances (e.g., pore forming moiety). The porous structure is believed to be formed by several mechanisms and their combinations. The dissipation, disbursement or diffusion of the solvent out of the solidifying flowable composition into the adjacent fluids may generate pores, including pore channels, within the polymer matrix. The infusion of aqueous medium, water or body fluid into the flowable composition also occurs and is in part also responsible for creation of pores. Generally, it is believed that the porous structure is formed during the transformation of the flowable composition to an implant, article and the like. During this process, it is believed, as explained above, that the organic solvent and thermoplastic polymer partition within the flowable composition into regions that are rich and poor in thermoplastic polymer. The partition is believed to occur as a result of the dynamic interaction of aqueous infusion and solvent dissipation. The infusion involves movement of aqueous medium, water or body fluid into the flowable composition and the dissipation involves movement of the organic solvent into the medium surrounding the flowable composition. The regions of the flowable composition that are poor in thermoplastic polymer become infused with a mixture of organic solvent and water, aqueous medium or body fluid. These regions are believed to eventually become the porous network of the implant, article and the like.

Typically, the macroscopic structure of the solid matrix involves a core and a skin. Typically, the core and skin are microporous but the skin pores are of smaller size than those of the core unless a separate pore forming agent is used as discussed below. Preferably, the outer skin portion of the solid matrix has pores with diameters significantly smaller in size than these pores in the inner core portion. The pores of the core are preferably substantially uniform and the skin is typically functionally non-porous compared to the porous nature of the core. The size of the pores of the implant, article, device and the like are in the range of about 4-1000 microns, preferably the size of pores of the skin layer are about 1-500 microns. The porosity of such matrices is described by U.S. Pat. No. 5,324,519, the disclosure of which is incorporated herein by reference.

The solid microporous implant, article, device and the like will have a porosity in the range of about 5-95% as measured by the percent solid of the volume of the solid. The development of the degree of porosity will be governed at least in part by the degree of water solubility of the organic solvent and thermoplastic polymer. If the water solubility of the organic solvent is high and that of the polymer is extremely low or non-existent, a substantial degree of porosity will be developed, typically on the order of 30 to 95%. If the organic solvent has a low water solubility and the polymer has a low to non-existent water solubility, a low degree of porosity will be developed, typically on the order of 5 to 40%. It is believed that the degree of porosity is in part controlled by the polymer-solvent partition when the flowable composition contacts an aqueous medium and the like. The control of the degree of porosity is beneficial for generation of differing kinds of biodegradable articles, implants and devices according to the invention. For example, if strength is a requirement for the article, implant or device and the like, it may be beneficial to have a low degree of porosity.

Solid Biodegradable Articles

Biodegradable drug delivery products can be prepared by the transformation process using water or an aqueous medium or body fluid to cause solidification. Generally, these products are ex vivo solid matrices. If the ex vivo solid matrix is to have a particular shape, it can be obtained by transforming the flowable composition in a suitable mold following the moldable implant precursor technique described above. After the precursor has been formed, it can be contacted with additional aqueous medium to complete the transformation. Alternatively, the flowable composition can be placed in a closed mold that is permeable to aqueous medium and the mold with composition can be contacted with aqueous medium such as be submerging in an aqueous bath. Preferably, the flowable composition in this instance will have a moderate to high viscosity.

Microcapsules and microparticles can be formed by techniques known in the art. Briefly, the microcapsule preparation involves formation of an emulsion of bioactive agent-carrier micelles in the flowable composition where the carrier is a nonsolvent for the biocompatible, biodegradable, branched thermoplastic polymer of the invention. The micelles are filtered and then suspended in an aqueous medium. The coating of flowable composition on the surfaces of the micelles then solidifies to form the porous microcapsules. Microparticles are formed in a similar process. A mixture of flowable composition and bioactive agent is added dropwise by spraying, dripping, aerosolizing or by other similar techniques to a nonsolvent for the flowable composition. The size and shape of the droplets is controlled to produce the desired shape and size of the porous microparticles. Sheets, membranes and films can be produced by casting the flowable composition onto a suitable nonsolvent and allowing the transformation to take place. Similarly, the viscosity of the flowable composition can be adjusted so that when sprayed or aerosolized, strings rather than droplets are formed. These strings can be cast upon a nonsolvent for the flowable composition such that a filamentous scaffold or membrane is produced. Also, suture material or other similar material can be formed by extrusion of the flowable composition into a non-solvent bath. The extrusion orifice will control the size and shape of the extruded product. The techniques for formation of these ex vivo solid matrices are described in U.S. Pat. Nos. 4,652,441; 4,917,893; 4,954,298; 5,061,492; 5,330,767; 5,476,663; 5,575,987; 5,480,656; 5,643,607; 5,631,020; 5,631,021; 5,651,990, the disclosures of which are incorporated herein by reference with the proviso that the polymers used are the biocompatible, biodegradable, thermoplastic polymers disclosed herein.

These ex vivo solid matrices can be used according to their known functions. Additionally, the implants and other solid articles are can be inserted in a body using techniques known to the art such as through an incision or by trocar.

The present invention also provides an implant. The implant includes a biodegradable, biocompatible thermoplastic polymer that is at least substantially insoluble in aqueous medium, water or body fluid; and a biological agent, a metabolite thereof, a biological agently acceptable salt thereof, or a prodrug thereof. The implant has a solid or gelatinous microporous matrix, wherein the matrix is a core surrounded by a skin. The implant can further include a biocompatible organic liquid, at standard temperature and pressure, in which the thermoplastic polymer is soluble. The amount of biocompatible organic liquid, if present, is preferably minor, such as from about 0 wt. % to about 20 wt. % of the composition. In addition, the amount of biocompatible organic liquid preferably decreases over time. The core preferably contains pores of diameters from about 1 to about 1000 microns. The skin preferably contains pores of smaller diameters than those of the core pores. In addition, the skin pores are preferably of a size such that the skin is functionally non-porous in comparison with the core. The implant can have any suitable shape and can have any suitable form. For example, the implant can be a solid, semi-solid, wax-like, viscous, or the implant can be gelatinous.

As used herein, “treating” or “treat” includes (i) preventing a pathologic condition (e.g., a solid tumor) from occurring (e.g. prophylaxis); (ii) inhibiting the pathologic condition (e.g., a solid tumor) or arresting its development; and (iii) relieving the pathologic condition (e.g., relieving the symptoms associated with a solid tumor).

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

“Therapeutically effective amount” is intended to include an amount of a biological agent, a metabolite thereof, a biological agently acceptable salt thereof, or a prodrug thereof useful in the present invention or an amount of the combination of biological agents, metabolites thereof, biological agently acceptable salts thereof, or prodrugs thereof, e.g., to treat or prevent the underlying disorder or disease, or to treat the symptoms associated with the underlying disorder or disease in a host. The combination of biological agents, metabolites thereof, biological agently acceptable salts thereof, or prodrugs thereof is preferably a synergistic combination. Synergy, as described for example by Chou and Talalay, Adv. Enzyme Regul. 22:27-55 (1984), occurs when the effect of the biological agents, metabolites thereof, biological agently acceptable salts thereof, or prodrugs thereof when administered in combination is greater than the additive effect of the biological agents, metabolites thereof, biological agently acceptable salts thereof, or prodrugs thereof when administered alone as a single agent. In general, a synergistic effect is most clearly demonstrated at suboptimal concentrations of the biological agents, metabolites thereof, biological agently acceptable salts thereof, or prodrugs thereof. Synergy can be in terms of lower cytotoxicity, increased activity, or some other beneficial effect of the combination compared with the individual components.

As used herein, “biological agently acceptable salts” refer to derivatives wherein the parent compound is modified by making acid or base salts thereof. Examples of biological agently acceptable salts include, but are not limited to, mineral or organic acid salts of basic residues such as amines; alkali or organic salts of acidic residues such as carboxylic acids; and the like. The biological agently acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, isethionic, and the like. Specifically, the biological agently acceptable salts can include those salts that naturally occur in vivo in a mammal.

The biological agently acceptable salts useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Biological agent Sciences, 17th ed., Mack Publishing Company, Easton, Pa., 1985, p. 1418, the disclosure of which is hereby incorporated by reference.

The phrase “biological agently acceptable” is employed herein to refer to those compounds (e.g., chemotherapeutic agents) which are, within the scope of sound medical judgment, suitable for use in contact with the tissues of human beings and animals without excessive toxicity, irritation, allergic response, or other problem or complication commensurate with a reasonable benefit/risk ratio.

Biological Agent Kits

The present invention provides biological agent kits. Such kits are suitable for in situ formation of a biodegradable implant in a body. The kits can include a first container that includes a flowable composition. The composition can include a biodegradable, biocompatible thermoplastic polymer that is at least substantially insoluble in aqueous medium, water or body fluid; and a biocompatible organic liquid at standard temperature and pressure, in which the thermoplastic polymer is soluble. The kit can also include a second container that includes a biological agent, a metabolite thereof, a biological agently acceptable salt thereof, or a prodrug thereof. The biological agent kit can further optionally include instructions or printed indicia for assembling and/or using the biological agent kit.

Specifically, the first container can include a syringe or a catheter; and the second container can independently include a syringe or a catheter. Additionally, the first container can include a syringe, the second container can include a syringe, and both syringes can be configured to directly connect to each other.

Specific Ranges, Values and Embodiments

In one specific embodiment of the present invention, the biodegradable, biocompatible thermoplastic polymer can have a formula incorporating monomeric units selected from the group of lactides, glycolides, caprolactones, glycerides, anhydrides, amides, urethanes, esteramides, orthoesters, dioxanones, acetals, ketals, carbonates, phosphazenes, hydroxybutyrates, hydroxyvalerates, alkylene oxalates, alkylene succinates, amino acids, and any combination thereof; and the formula contains the monomeric units random or block order.

In another specific embodiment of the present invention, the biodegradable, biocompatible thermoplastic polymer can be a polymer or copolymer of lactide monomeric units, caprolactone monomeric units, glycolide monomeric units, or any combination thereof.

In another specific embodiment of the present invention, the biodegradable, biocompatible thermoplastic polymer can include a polymer selected from the group of polylactides, polyglycolides, polycaprolactones, polydioxanones, polycarbonates, polyhydroxybutyrates, polyalkyene oxalates, polyanhydrides, polyamides, polyesteramides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, poly(methyl vinyl ether), polyesters, polyalkylglycols, copolymers thereof, block copolymers thereof, terpolymers thereof, combinations thereof, and mixtures thereof.

In another specific embodiment of the present invention, the biodegradable, biocompatible thermoplastic polymer can include at least one polyester.

In another specific embodiment of the present invention, the biodegradable, biocompatible thermoplastic polymer can be at least one of a polylactide, a polyglycolide, a polycaprolactone, a copolymer thereof, a terpolymer thereof, or any combination thereof.

In another specific embodiment of the present invention, the biodegradable, biocompatible thermoplastic polymer can be a poly(DL-lactide-co-glycolide). In another specific embodiment of the present invention, the biodegradable, biocompatible thermoplastic polymer can be a poly(DL-lactide-co-glycolide) having a carboxy terminal group. In another specific embodiment of the present invention, the biodegradable, biocompatible thermoplastic polymer can be a poly(DL-lactide-co-glycolide) without a carboxy terminal group. In another specific embodiment of the present invention, the biodegradable, biocompatible thermoplastic polymer can be 50/50 poly(DL-lactide-co-glycolide) having a carboxy terminal group. In another specific embodiment of the present invention, the biodegradable, biocompatible thermoplastic polymer can be 75/25 poly(DL-lactide-co-glycolide) without a carboxy terminal group.

In another specific embodiment of the present invention, the biodegradable, biocompatible thermoplastic polymer can be present in up to about 80 wt. % of the composition. In another specific embodiment of the present invention, the biodegradable, biocompatible thermoplastic polymer can be present in more than about 10 wt. % of the composition. In another specific embodiment of the present invention, the biodegradable, biocompatible thermoplastic polymer can be present in about 10 wt. % to about 80 wt. % of the composition. In another specific embodiment of the present invention, the biodegradable, biocompatible thermoplastic polymer can be present in about 30 wt. % to about 50 wt. % of the composition.

In another specific embodiment of the present invention, the biodegradable, biocompatible thermoplastic polymer can have an average molecular weight of more than about 15,000. In another specific embodiment of the present invention, the biodegradable, biocompatible thermoplastic polymer can have an average molecular weight of up to about 45,000. In another specific embodiment of the present invention, the biodegradable, biocompatible thermoplastic polymer can have an average molecular weight of about 15,000 to about 45,000.

In one embodiment of the present invention, the biocompatible organic liquid can have a water solubility ranging from completely insoluble in any proportion to completely soluble in all proportions. In another embodiment of the present invention, the biocompatible organic liquid can be completely insoluble in water but will diffuse into body fluid. In another embodiment of the present invention, the biocompatible organic liquid can be at least partially water-soluble. In another embodiment of the present invention, the biocompatible organic liquid can be completely water-soluble. In another embodiment of the present invention, the biocompatible liquid can be dispersible in aqueous medium, water, or body fluid.

In another embodiment of the present invention, the biocompatible organic liquid can be a polar protic liquid. In another embodiment of the present invention, the biocompatible organic liquid can be a polar aprotic liquid.

In another embodiment of the present invention, the biocompatible organic liquid can be a cyclic, aliphatic, linear aliphatic, branched aliphatic or aromatic organic compound, that is liquid at ambient and physiological temperature, and contains at least one functional group selected from the group of alcohols, ketones, ethers, amides, amines, alkylamines, esters, carbonates, sulfoxides, sulfones, and sulfonates.

In another embodiment of the present invention, the biocompatible organic liquid can be selected from the group of substituted heterocyclic compounds, esters of carbonic acid and alkyl alcohols, alkyl esters of monocarboxylic acids, aryl esters of monocarboxylic acids, aralkyl esters of monocarboxylic acids, alkyl esters of dicarboxylic acids, aryl esters of dicarboxylic acids, aralkyl esters of dicarboxylic acids, alkyl esters of tricarboxylic acids, aryl esters of tricarboxylic acids, aralkyl esters of tricarboxylic acids, alkyl ketones, aryl ketones, aralkyl ketones, alcohols, polyalcohols, alkylamides, dialkylamides, alkylsulfoxides, dialkylsulfoxides, alkylsulfones, dialkylsulfones, lactones, cyclic alkyl amides, cyclic alkyl amines, aromatic amides, aromatic amines, mixtures thereof, and combinations thereof.

In another embodiment of the present invention, the biocompatible organic liquid can be selected from the group of N-methyl-2-pyrrolidone, 2-pyrrolidone, (C₂-C₈)aliphatic alcohol, glycerol, tetraglycol, glycerol formal, 2,2-dimethyl-1,3-dioxolone-4-methanol, ethyl acetate, ethyl lactate, ethyl butyrate, dibutyl malonate, tributyl citrate, tri-n-hexyl acetylcitrate, diethyl succinate, diethyl glutarate, diethyl malonate, triethyl citrate, triacetin, tributyrin, diethyl carbonate, propylene carbonate, acetone, methyl ethyl ketone, dimethylacetamide, dimethylformamide, caprolactam, dimethyl sulfoxide, dimethyl sulfone, tetrahydrofuran, caprolactam, N,N-diethyl-m-toluamide, 1-dodecylazacycloheptan-2-one, 1,3-dimethyl-3,4,5,6-tetrahydro-2-(1H)-pyrimidinone, benzyl benzoate, and combinations thereof.

In another embodiment of the present invention, the biocompatible organic liquid can have a molecular weight in the range of about 30 to about 500.

In another embodiment of the present invention, the biocompatible organic liquid can be N-methyl-2-pyrrolidone, 2-pyrrolidone, N,N-dimethylformamide, dimethyl sulfoxide, propylene carbonate, caprolactam, triacetin, or any combination thereof. In another embodiment of the present invention, the biocompatible organic liquid can be N-methyl-2-pyrrolidone.

In another embodiment of the present invention, the biocompatible liquid can be present in more than about 40 wt. % of the composition. In another embodiment of the present invention, the biocompatible liquid can be present in up to about 80 wt. % of the composition. In another embodiment of the present invention, the biocompatible liquid can be present in about 50 wt. % to about 70 wt. % of the composition.

### EXAMPLES

Sustained-Release of Drugs to the Eye Using the ATRIGEL® Delivery System

Introduction to the ATRIGEL® Drug Delivery Technology

QLT USA, a subsidiary of QLT, Inc. has developed a liquid, biodegradable drug delivery system (ATRIGEL®) for the sustained-release of small molecules, peptides and proteins. The delivery system consists of biodegradable polymers such as the lactide/glycolide copolymers dissolved in biocompatible solvents. A drug is incorporated into this solution and the resulting mixture is injected subcutaneously using standard syringes and needles. Upon contact with body fluids, the ATRIGEL® Delivery System solidifies and traps the drug in a solid implant. Drug is released at a predetermined rate as the implant undergoes biodegradation.

Using the ATRIGEL® Delivery System, Atrix has delivered a variety of drugs, ranging from small molecules to recombinant biopharmaceuticals with a duration of drug delivery ranging from 1 week to 6 months. Currently, Atrix has a number of FDA approved products on the market that utilize the ATRIGEL® Delivery System, including dental (ATRIDOX® ATRISORB® and ATRISORB®-D) and pharmaceutical products (ELIGARD® 7.5 mg, ELIGARD® 22.5 mg and ELIGARD® 30 mg) and several in clinical trials.

Advantages of the ATRIGEL® Delivery System

The ATRIGEL® Delivery System offers a number of distinct advantages over other parenteral sustained-release delivery systems. For example, microspheres must be manufactured using aseptic processes that may include the use of halogenated solvents. Furthermore, the drug to microsphere ratio is controlled by the encapsulation efficiency, a process that can result in the irretrievable loss of 25 to 50% of the API during the manufacture of the drug product. In comparison, the ATRIGEL® Delivery System is composed of biocompatible ingredients and is prepared by dissolving the appropriate biodegradable polymer in a biocompatible solvent. Unlike microspheres, the ATRIGEL® Delivery System can be terminally sterilized using conventional techniques, including gamma irradiation. The unique manufacturing process and proprietary product configuration essentially eliminates the loss of drug during manufacture. Furthermore, the ATRIGEL® Delivery System can deliver large doses of API in small injection volumes as compared to small doses in large injection volumes for microspheres. Most importantly, the ATRIGEL® depot protects sensitive biopharmaceuticals from in vivo degradation and enzymatic inactivation.

The ATRIGEL® technology is a patient-friendly delivery platform when compared to implantable or reservoir devices. The ATRIGEL® drug product is injected subcutaneously and the resulting implant releases drug over a predetermined interval of time. Typically, the implant biodegrades at the same rate that the drug is released; therefore, the injection site essentially resolves in time for the next injection. In comparison, mechanical implants must be removed surgically and replaced or refilled after the drug reservoir is depleted.

When used to administer a biological agent to the eye, the ATRIGEL® Delivery System employs substances in an effective and suitable amount, to diminish the occurrence and/or severity of irritation to the eye and surrounding tissue.

### Example 1

Tolerability of the ATRIGEL® Delivery System Following Intraocular Injection

A series of preclinical studies were conducted to determine the tolerability of the ATRIGEL® Delivery System following intraocular administration. In these studies, New Zealand White rabbits were injected with one of three ATRIGEL® vehicles. Injections were performed directly into the eye (intravitreal injection), under the conjunctiva (subconjunctival injection) or through the membrane covering the muscles and nerves at the back of the eyeball (subtenon injection). The rabbits were observed periodically over 28 days for local reactions and ocular acuity. In addition, the vitreous humor was sampled to assess the cytopathological affect of each ATRIGEL® vehicle.

As expected with any intraocular administration, mild conjunctival congestion was noted for all ATRIGEL® solutions; however, this transient response resolved within 72 hours. Intraocular pressure and visual acuity remained unchanged throughout the study. Cytopathological assessment of the vitreous humor at Days 3, 14 and 28 post dosing showed that the white blood cell count (WBCs) and protein levels were all normal. In addition, no inflammatory or atypical cells or infectious agents were noted in any treated eye at any time post dosing.

These results demonstrate that the ATRIGEL® Delivery System is well tolerated and appears to be inert following intraocular injection. In fact, ATRIGEL® drug products may attenuate the local response of certain drugs. For example, in a subsequent study, the tolerability of a formulation prepared by mixing an ATRIGEL® vehicle with a known ocular irritant (benzethonium chloride) was compared to the tolerability of an aqueous solution of the same material. Gross observations and cytopathological evaluations demonstrated that the irritant alone produced marked conjunctival swelling, severe aqueous and cellular flare and almost complete loss of the transparency of the cornea one day after intravitreal injection. However, the ATRIGEL®/irritant formulation showed only mild to moderate conjunctival swelling, moderate aqueous and cellular flare with no loss in transparency of the cornea over the same dosing period. Thus, the slow-release character of the ATRIGEL® depot exposes sensitive ocular tissue to lower levels of the irritant and thereby minimizes the probability of a local adverse event.

In Conclusion, the ATRIGEL® Delivery System is well suited for the sustained delivery of therapeutic agents to the eye.

### Example 2

Several ATRIGEL® formulations containing PEG300, mPEG350, PEG400, NMP, triacetin, DMSO as well as neat DMSO and an aqueous solution of BEC were evaluated either intravitreally or subconjuctivally over three days in the rabbit eye. Several ATRIGEL® formulations were found to be acceptable for ocular implantation over a short time period using either route of administration, specifically, these included formulations containing PEG300, mPEG350, PEG400 and NMP. Therefore, a long-term irritation study was conducted with ATRIGEL® formulations containing PEG300, mPEG350 and NMP utilizing both routes of administration. The results of the long-term study show that polymer degradation occurs as expected and that no prolonged irritation is observed. Thus, ATRIGEL® formulations containing PEG300, mPEG350 and NMP can be considered acceptable vehicles for intravitreal or subconjuctival implantation and subsequent drug delivery.

The objective of this project is to assess the feasibility of the ATRIGEL® delivery system as an extended release drug delivery vehicle to the eye. ATRIGEL® vehicles will be subjected to injection in various locations in and around the eye with the ultimate purpose of the project to identify vehicles and injection techniques that are clinically acceptable and form implants that do not interfere with the function of the eye or cause significant tissue reaction. If this preliminary phase of work is successful, subsequent proposals will be generated to evaluate drug delivery to the eye.

A series of preclinical studies in rabbits will investigate various injection techniques and locations with a range of ATRIGEL® vehicles. The tissue reaction at the injection sites and the various structures of the eye will also be evaluated. The injection sites will include subconjuctival injection, which are injections against the outside of the eye and intravitreal injections through the sclera (the tough outer membrane) of the eye. This will hopefully result in an implant that is affixed to the sclera and forms a plug to prevent loss of vitreous humor. An intravitreally injected implant has the advantage of direct contact with the interior of the eye, thereby allowing the most efficient delivery of drug. However, this route of administration has a significantly higher potential for adverse effects.

The initial studies investigating these injection techniques and locations will be performed with small numbers of rabbits that will be sacrificed after 72 hours. Once the initial studies are complete and acceptable ATRIGEL® formulations are identified, a long-term irritation study will be conducted. In all studies the rabbits will be observed closely for adverse effects and euthanized if appropriate. Slit lamp observations to assess anterior chamber features will be graded on a numerical scale using a modified McDonald-Shadduck scoring system. Histology of the injection sites and of key tissues of the eye, particularly the retina, and cytopathology of the vitreous humor will also be evaluated. The cytopathology report will include white blood cell count, protein count and specific gravity values.

Due to the sensitivity of the tissues in the eye, only ATRIGEL® vehicles with the most biocompatible solvents will be used in the initial studies. The initial solvents studied will consist of polyethylene glycol 300 (PEG300), PEG400, polyethylene glycol monomethylether 350 (mPEG350), n-methylpyrrolidone (NMP), dimethylsulfoxide (DMSO), and glycerol triacetate (triacetin). In addition, a known ocular irritant, benzethonium chloride (BEC) will be evaluated to observe a positive response. A single polymer, 50/50 poly(lactide-co-glycolide) (PLGH) with an inherent viscosity of 0.18 dL/g will be used throughout the studies, a constant injection volume of 50 μL and a 25-gauge ⅝ inch needle will also be used.

4.1. ATRS917

The first in-vivo rabbit study was completed on Jun. 19, 2003 and it evaluated the intravitreal route of injection with 6 ATRIGEL® vehicle formulations. A Vicryl biodegradable suture was used as the control test article. The ATRIGEL® formulations are listed below:

| | | | Injection | Dose | |
|---|---|---|---|---|---|
| Group | No. | Formulation | Location | Vol. | Euthanasia |
| A | 3 | 15% 50/50 PLGH | Intravitreal | 50 μL | Day 3 |
| | | 0.18 in PEG300 | | | |
| B | 3 | 25% 50/50 PLGH | Intravitreal | 50 μL | Day 3 |
| | | 0.18 in PEG300 | | | |
| C | 3 | 15% 50/50 PLGH | Intravitreal | 50 μL | Day 3 |
| | | 0.18 in mPEG350 | | | |
| D | 3 | 25% 50/50 PLGH | Intravitreal | 50 μL | Day 3 |
| | | 0.18 in mPEG350 | | | |
| E | 3 | 15% 50/50 PLGH | Intravitreal | 50 μL | Day 3 |
| | | 0.18 in PEG400 | | | |
| F | 3 | 25% 50/50 PLGH | Intravitreal | 50 μL | Day 3 |
| | | 0.18 in PEG400 | | | |

The PEG400 formulations (Groups E and F) were the most viscous and somewhat hard to inject through the 25-gauge needle, however all the injections went smoothly with no difficulties. At 24 hours post injection there was no irritation associated with the treated eyes or ophthalmic abnormalities noted for Groups A-D. In Groups E and F, one-third of the treated eyes showed conjunctival discharge with no other abnormalities observed. At 72 hours post injection no irritation or ophthalmic abnormalities were noted for Groups A, D and E however, Groups B, C and F showed one out of three eyes having mild aqueous or cellular flare with no other abnormalities noted. No pupillary response was noted in the animals due to pharmacological blockage associated with the tropicamide pupil dilation solution used to help grade the posterior portion of the eye. This result will be expected in all future studies as well, and the lack of pupil response is not associated with the ATRIGEL® implants.

Ocular pressure, specific gravity, white blood cell and protein counts were all at normal levels and no inflammatory, atypical cells or infectious agents were observed in any of the treated eyes. The intravitreal injections were very clean and the puncture hole self-sealed with ATRIGEL® when the needle was removed from the eye. Necropsy showed the implants to be attached to the inner surface of the eye and not floating in the vitreous humor.

The results of this study suggest that ATRIGEL® PEG and mPEG formulations are well tolerated when injected in the eye. No significant ocular/tissue irritation was observed for any test article. The only concern the ophthalmologists, Biological Test Center (BTC) Labs, conducting the study had was that the injection size was somewhat large for a solid depot implant. They felt the sight of the rabbit was impaired using this injection volume. Since the size of the implant was not the foremost concern of this study, but ocular and tissue irritation was, we did not optimize the injection volume. This concern will be addressed as further development continues.

4.2. ATRS929

A second in-vivo rabbit study was completed on Aug. 20, 2003. Four ATRIGEL® formulations via intravitreal injection and two formulations via subconjuctival injection were evaluated. A 7-0 Vicryl biodegradable suture was again used as the control test article. The ATRIGEL® formulations are listed below:

| | | | Injection | Dose | |
|---|---|---|---|---|---|
| Group | No. | Formulation | Location | Vol. | Euthanasia |
| A | 3 | 25% 50/50 PLGH | Intravitreal | 50 μL | Day 3 |
| | | 0.18 in NMP | | | |
| B | 3 | 35% 50/50 PLGH | Intravitreal | 50 μL | Day 3 |
| | | 0.18 in NMP | | | |
| C | 3 | 15% 50/50 PLGH | Intravitreal | 50 μL | Day 3 |
| | | 0.18 in Triacetin | | | |
| D | 3 | 25% 50/50 PLGH | Intravitreal | 50 μL | Day 3 |
| | | 0.18 in Triacetin | | | |
| E | 3 | 25% 50/50 PLGH | Subconjunctival | 50 μL | Day 3 |
| | | 0.18 in PEG300 | | | |
| F | 3 | 25% 50/50 PLGH | Subconjunctival | 50 μL | Day 3 |
| | | 0.18 in PEG400 | | | |

Groups A-D were injected intravitreally and Groups E and F subconjuctivally. (Note: For reference, Groups E and F formulations were also evaluated intravitreally in ATRS917.) According to BTC Labs all the injections went smoothly with no difficulties.

At 24 hours post injection most animals exhibited a mild to moderate conjunctival congestion and swelling in the treated eyes (left eyes). Aqueous and cellular flare was noted in three of the treated eyes (two eyes in Group A and one eye in Group C). Nuclear cataracts were noted in three of the treated eyes (two eyes in Group C and one eye in Group D). In Groups C and D (triacetin ATRIGEL® formulations) the test article enveloped the lens anteriorly and posteriorly, and migrated to the lens nucleus. Three of the treated eyes and one control eye (Group A) were noted to have a few scattered opacities in the vitreous chamber. No other abnormal ocular observations were noted.

At 72 hours post injection only one animal, Group A, exhibited a mild conjunctival congestion in the treated eye. No aqueous or cellular flare was noted after 72 hours and only one eye was noted to have a nuclear cataract in the treated eye of a Group C animal. The test articles in two of the treated eyes of the Group C animals were located in the inferior part of the globe of the posterior segment; the test articles were conical in shape. In one animal, small 1 to 2 mm segments of the test article migrated to the peripapillary region of the optic nerve head. One treated eye (Group D) was observed to have a mild choroidal/retinal inflammation.

As with the initial ocular ATRIGEL® study (ATRS917) the injections were very clean and the puncture hole self-sealed with ATRIGEL® when the needle was removed from the eye. Necropsy showed the implants in groups A and B to be attached to the inner surface of the eye and not floating in the vitreous humor. Group C and D implants were found associated with the lens and exhibited a very thin film-like morphology. Groups E and F implants were found adhered to the outer surface of the eye. Specific gravity, ocular pressure, white blood cell and protein counts were all at normal levels for all formulations investigated. However, one animal in Group C was found to have a low number of inflammatory cells.

The results of this study suggest that triacetin would not be an acceptable carrier solvent for an ocular ATRIGEL® implant. However, the NMP formulation showed acceptable results which are comparable to the intravitreally injected PEG300 and 400 studied in the first in-vivo (ATRS917) evaluation, that is, similar cytopathology and ocular observations were noted. The low ocular/tissue irritation of PEG300 and 400 implants, which were injected subconjuctivally and adhered to the outer surface of the eye, is also encouraging and gives additional flexibility of the ATRIGEL® system as an ocular drug delivery device.

4.3. ATRS939

A third in-vivo rabbit study was completed on Sep. 23, 2003. Four ATRIGEL® formulations via intravitreal injection and two formulations via subconjuctival injection were evaluated. As with the previous two in-vivo studies, a 7-0 Vicryl biodegradable suture was used as the control test article. The ATRIGEL® formulations are listed below:

| | | | Injection | Dose | |
|---|---|---|---|---|---|
| Group | No. | Formulation | Location | Vol. | Euthanasia |
| A | 3 | Dimethylsulfoxide (DMSO) | Intravitreal | 50 μL | Day 3 |
| B | 3 | 40% 50/50 PLGH 0.18 in DMSO | Intravitreal | 50 μL | Day 3 |
| C | 3 | 2% BEC in H₂O | Intravitreal | 50 μL | Day 3 |
| D | 3 | 2% BEC in 25% 50/50 PLGH 0.18 in PEG300 | Intravitreal | 50 μL | Day 3 |
| E | 3 | 25% 50/50 PLGH 0.18 in NMP | Subconjunctival | 50 μL | Day 3 |
| F | 3 | 25% 50/50 PLGH 0.18 in triacetin | Subconjunctival | 50 μL | Day 3 |

Groups A-D were injected intravitreally and Groups E and F subconjuctivally. (Note: For reference, Groups E and F formulations were also evaluated intravitreally in the ATRS929.) According to BTC Labs all the injections went smoothly with no difficulties.

At 24 hours post injection one animal in Groups A and E exhibited mild conjunctival congestion. All animals in Groups C, D and F showed at least conjunctival congestion, which was bright red in color with accompanying perilimbal injection covering at least 75% of the circumference of the perilimbal region. Conjunctival swelling in Group C was also pronounced and in Group F was mild. In addition to the abnormalities seen in Group C was also the almost complete loss of the transparency of the cornea with ˜76-100% surface involvement. Group C also showed severe aqueous and cellular flare. Group C and D exhibited minimal to moderate injection of the tertiary vessels of the iris with slight swelling of the iris stroma, in addition, many opacities and marked blurring of the fundus details was observed in the vitreous as well as mild to moderate choroidal/retinal inflammation. No other observations were noted for Groups A, B, E and F at 24 hours post injection.

At 72 hours post injection all animals in Groups B, E and F showed no abnormal ocular observations besides lack of pupillary response, which was also expected. One animal in Group A showed mild retinal hemorrhage and inflammation. The animals in Group C and D still showed mild to moderate conjunctival congestion, with Group C animals also showing discharge and swelling. Group C animals also exhibited cornea transparency loss, iris involvement, nuclear and mature cataracts and opacities which caused marked blurring of the fundus details. The retinal detachment, hemorrhage and inflammation could not be evaluated in Groups C and D.

The cytopathological findings, conducted on the vitreous humor of Groups A-D, indicated that the specific gravity and protein levels were elevated in two-thirds of the animals in Groups C and D. Significant inflammation was observed in all animals in Groups C and D and one animal from Group A. All retinal cells were found to be normal in appearance and no atypical cells or infectious agents were observed.

As with the initial ocular ATRIGEL® studies (ATRS917 and 939) the injections were very clean and the puncture hole self-sealed with ATRIGEL® when the needle was removed from the eye. Necropsy showed that Group B, which contained 40% polymer, contained a much larger implant than Group D, which only contained 25% polymer. This is partially due to the swelling of the polymer upon solidification as well as the polymer concentration itself and one would expect the higher polymer concentration to produce a larger implant. These intravitreally injected ATRIGEL® implants were found to be associated with the side of the eye and it was unclear if they were “anchored” to the eye. Groups E and F implants were found adhered to the outer surface of the eye and had a flat, disc-like morphology as compared to the intravitreally injected implants, which were spherical in shape.

The results of this study suggest that BEC did cause significant ocular irritation, as expected, and that BEC in ATRIGEL® (Group D) did attenuate the cellular flare, conjunctival swelling, discharge and congestion, but did not decrease the actual inflammation in the vitreous humor. The NMP formulation exhibited the least irritation out of the entire set of test articles investigated and triacetin caused significant conjunctival congestion. The DMSO formulation and neat solvent were found not to show inflammation above test articles investigate in the first and second in-vivo studies (ATRS 917 and 929).

5. 28-Day ATRIGEL® Feasibility Study, ATRS948

The fourth in-vivo rabbit study was initiated on Oct. 28, 2003. The study evaluated intravitreal and subconjuctival routes of injection with three ATRIGEL® vehicle formulations over a period of 28 days. This study was undertaken to evaluate the long-term irritation of ocular ATRIGEL® implants as well as to investigate the degradation kinetics of the implants. The ATRIGEL® formulations are listed below:

| | | | Injection Location | Dose | |
|---|---|---|---|---|---|
| Group | No. | Formulation | (Both Eyes) | Volume | Euthanasia |
| A | 2 | 25% 50/50 PLGH 0.18 in PEG300 | Intravitreal | 50 μL | Day 14 |
| B | 2 | 25% 50/50 PLGH 0.18 in PEG300 | Intravitreal | 50 μL | Day 28 |
| C | 2 | 25% 50/50 PLGH 0.18 in PEG300 | Subconjunctival | 50 μL | Day 14 |
| D | 2 | 25% 50/50 PLGH 0.18 in PEG300 | Subconjunctival | 50 μL | Day 28 |
| E | 2 | 35% 50/50 PLGH 0.18 in mPEG350 | Intravitreal | 50 μL | Day 14 |
| F | 2 | 35% 50/50 PLGH 0.18 in mPEG350 | Intravitreal | 50 μL | Day 28 |
| G | 2 | 35% 50/50 PLGH 0.18 in mPEG350 | Subconjunctival | 50 μL | Day 14 |
| H | 2 | 35% 50/50 PLGH 0.18 in mPEG350 | Subconjunctival | 50 μL | Day 28 |
| I | 2 | 45% 50/50 PLGH 0.18 in NMP | Intravitreal | 50 μL | Day 14 |
| J | 2 | 45% 50/50 PLGH 0.18 in NMP | Intravitreal | 50 μL | Day 28 |
| K | 2 | 45% 50/50 PLGH 0.18 in NMP | Subconjunctival | 50 μL | Day 14 |
| L | 2 | 45% 50/50 PLGH 0.18 in NMP | Subconjunctival | 50 μL | Day 28 |

Groups A-B, E-F, and I-J were injected intravitreally and Groups C-D, G-H, and K-L subconjuctivally. According to BTC Labs all the injections went smoothly with no difficulties.

At 24 hours post injection most animals in Groups A, C, D, E, H and K exhibited mild conjunctival congestion and mild conjunctival swelling was also observed in animals from Groups C, D and E. In addition, one animal in Group D exhibited an abundant amount of conjunctival discharge. Aqueous flare was noted in one animal from Group C and cellular flare was noted in one animal from Group A and C. Iris involvement was noted in one animal from Group A. No other abnormal ocular observations were noted.

One week after implantation only one abnormal ocular observation was noted. This involved the slight loss of transparency of the cornea in one animal from Group D. The underlying structures of the eye were still clearly visible although some cloudiness was apparent encompassing 1-25% of the cornea. This abnormal observation was found to be due to the animal scratching its eye, not from the ATRIGEL® test article.

Examination timepoints of two, three and four weeks post implantation did not reveal any abnormal observations. However, one animal in Group J appeared to have its lens slightly pushed forward. In addition, cyopathological findings, conducted on the vitreous humor of Groups A-B, E-F, and I-J, indicate that specific gravity, ocular pressure, white blood cell and protein counts were all at normal levels for these intravitreally injected formulations. In addition, no atypical or inflammatory cells were observed.

Necropsy of selected eyes was accomplished to assess polymer degradation and implant morphology on Days 14 and 28. On Day 14, Group A, E and I implants were found to be soft, jelly-like and semitransparent structures. These intravitreally injected ATRIGEL® implants were found to be associated with the side of the eye and it was unclear if they were “anchored” to the eye. Group C, G and K implants were found to be adhered to the outside of the eye, show integrity and exhibit signs of degradation. By Day 28, only one implant was found corresponding to Group B, this implant was very soft, semitransparent and obviously degraded. No other implants were found on Day 28 and no signs that an implant was previously present were observed.

The results of this study show that similar 24-hour observations are observed as those in the first three short-term ocular ATRIGEL® evaluation studies. These observations are mostly limited to conjunctival congestion, which is a typical reaction to intravitreal or subconjuctival injections. No prolonged irritation or abnormal cytopathology was observed up to 28-Days post implantation. Necropsy revealed that implants found on Day 14 were obviously degraded and only one implant was found from the Day 28 timepoint. This result is very encouraging since complete degradation of the polymer is anticipated within this timeframe. In addition, the absence of irritation through Day 28 suggests that the degradation products of the ATRIGEL® do not cause irritation and are cleared from the eye.

5.2. ATRS1012

The fifth in-vivo rabbit study was initiated oil Aug. 18, 2004. The study evaluated the sub-Tenon's route of injection with three ATRIGEL® vehicle formulations over a period of 28 days. This study was undertaken to evaluate the long-term irritation of ocular ATRIGEL® implants as well as to investigate the degradation kinetics of the implants. The ATRIGEL® formulations are listed below:

| | | | Injection | Dose | |
|---|---|---|---|---|---|
| | | | Location | Vol- | Eutha- |
| Group | No. | Formulation | (Both Eyes) | ume | nasia |
| A | 2 | 25% 50/50 PLGH 0.18 in | Sub-Tenon's | 50 μL | Day 3 |
| | | PEG300 | | | |
| B | 2 | 25% 50/50 PLGH 0.18 in | Sub-Tenon's | 50 μL | Day 14 |
| | | PEG300 | | | |
| C | 2 | 25% 50/50 PLGH 0.18 in | Sub-Tenon's | 50 μL | Day 28 |
| | | PEG300 | | | |
| D | 2 | 30% 50/50 PLGH 0.18 in | Sub-Tenon's | 50 μL | Day 3 |
| | | mPEG350 | | | |
| E | 2 | 30% 50/50 PLGH 0.18 in | Sub-Tenon's | 50 μL | Day 14 |
| | | mPEG350 | | | |
| F | 2 | 30% 50/50 PLGH 0.18 in | Sub-Tenon's | 50 μL | Day 28 |
| | | mPEG350 | | | |
| G | 2 | 45% 50/50 PLGH 0.18 in | Sub-Tenon's | 50 μL | Day 3 |
| | | NMP | | | |
| H | 2 | 45% 50/50 PLGH 0.18 in | Sub-Tenon's | 50 μL | Day 28 |
| | | NMP | | | |
| I | 2 | 45% 50/50 PLGH 0.18 in | Sub-Tenon's | 50 μL | Day 14 |
| | | NMP | | | |

On Days 1 and/or 3, conjunctival congestion was exhibited in 17 of 36 eyes. Conjunctival congestion was exhibited by 6 of 12 eyes dosed with 25% 50/50 PLGH 0.18 in PEG300, 7 of 12 eyes dosed with 30% 50/50 PLGH 0.18 in mPEG350, and 4 of 12 eyes dosed with 45% 50/50 PLGH 0.18 in NMP. One of these eyes, dosed with 25% 50/50 PLGH 0.18 in PEG300, also exhibited conjunctival swelling on Day 1. One eye dosed with 30% 50/50 PLGH 0.18 in mPEG350 exhibited conjunctival discharge on Day 3; this eye was not observed to have conjunctival congestion during the study. Two eyes dosed with 45% 50/50 PLGH 0.18 in NMP exhibited some loss of corneal transparency near the conjunctival injection site; this observation occurred only on the day following injection (Day 1).

On Days 1, 3, 7, and/or 14, test article was observed to have leaked out or dislocated from the injection site in 9 of 36 eyes. Test article leakage or dislocation was observed in 1 of 12 eyes dosed with 25% 50/50 PLGH 0.18 in PEG300, 3 of 12 eyes dosed with 30% 50/50 PLGH 0.18 in mPEG350, and 5 of 12 eyes dosed with 45% 50/50 PLGH 0.18 in NMP. For these eyes, test article was present in the conjunctival area, the cornea surface, and/or the third eyelid.

At Day 21, all remaining eyes dosed with one of the two PEG test article formulations were observed to have only a trace amount of test article present; test article was clearly present, with a normal vascular response over the sites, in all remaining eyes dosed with the NMP formulation.

Cytopathological findings, conducted on the vitreous humor of all Groups, indicate that specific gravity, ocular pressure, white blood cell and protein counts were all at normal levels for these injected formulations. In addition, no atypical or inflammatory cells were observed. In the opinion of the consulting pathologist, fluid cytology findings were consistent with normal vitreous humor.

Necropsy of selected eyes was accomplished to assess polymer degradation and implant morphology on Days 3, 14 and 28. On Day 3, implants were found adhered to the sclera of the eye and were firm. On Day 14, implants were found to be adhered to the outside of the eye, show integrity but exhibit signs of degradation (softness). No implants were found on Day 28 and no signs that an implant was previously present were observed.

The results of this study show that similar 24-hour observations are observed as those in the first four ocular ATRIGEL® evaluation studies that evaluated intravitreal and subconjunctival routes of administration. These observations are mostly limited to conjunctival congestion, which is a typical reaction to intravitreal, subconjuctival or sub-Tenon's injections. No prolonged irritation or abnormal cytopathology was observed up to 28-Days post implantation. Necropsy revealed that implants found on Day 14 were slightly degraded and, as expected, no implants were found from the Day 28 timepoint. The absence of irritation through Day 28 suggests that the sub-Tenon's capsule accepts and tolerates an ATRIGEL® implant.

6. Discussion

The results of the first three, short-term, in-vivo ocular feasibility studies suggest that PEG300, PEG400, mPEG350 and NMP would be suitable carrier solvents for either intravitreal or subconjuctival ATRIGEL® implantation. These carrier solvents showed minimal ocular and tissue irritation over a 3-day period using either injection route. The DMSO ATRIGEL® formulation did not show irritation above previous test articles that were evaluated intravitreally and could possibly be tolerated subconjuctival, however, the biocompatibility of DMSO in questionable. Furthermore, triacetin was found not he compatible with ocular implantation due to poor implant formation as well as irritation issues.

Knowing that PEG300, mPEG350 and NMP ATRIGEL® formulations were compatible with ocular implantation over 3-Days, two long-term irritation studies were completed with these ATRIGEL® vehicles. The results of the long-term irritation studies indicate that no significant irritation is present over the 28-Day period for intravitreal, subconjuctival or sub-Tenons injected implants. Furthermore, the absence of ATRIGEL® implants upon completion of the study reveals that ATRIGEL® degradation proceeds as expected and that the eye does not trap the degradation products.

The study results also indicate that intravitreally injected implants are associated with the inner surface of the eye and do not float in the vitreous humor. The necropsy of intravitreally injected eyes suggests that the self-sealing of the injection hole with ATRIGEL® causes the rest of the implant to be “anchored” to the inner surface of the eye, which would restrict the implant from moving about the vitreous humor causing vision impairment. Similarly, the subconjuctivally and sub-Tenons injected implants adhere to the outer surface of the eye due to the tackiness of the ATRIGEL® implant. This implies that mass transport of drug through the outer membrane of the eye would be increased due to surface contact of the implant with the eye. The indicated acceptability of the subconjuctival and sub-Tenon injection routes also increases the flexibility of the ATRIGEL® delivery system since the injection volume, polymer concentration or drug load could be increased to meet the needs of a longer-duration delivery period.

Summary: A series of animal studies were conducted to determine the tolerability of the ATRIGEL® Delivery System following injection in and around the eye. In these studies, rabbits were injected with one of several ATRIGEL® solutions. Injections were performed directly into the eye (intravitreal injection), under the conjunctiva (subconjunctival injection) or through the membrane covering the muscles and nerves at the back of the eyeball (subtenon injection). The rabbits were observed periodically for local reactions and for the loss or impairment of vision. In addition, the fluid in the eye was analyzed for any indication of damage.

As expected with the injection of any material into the eye, minimal redness was noted for all ATRIGEL® solutions; however, this redness disappeared within 72 hours. The pressure within the eye remained unchanged throughout the study. More importantly, vision was not impaired. Evaluation of the fluid within the eye under a microscope showed that the white blood cell count (WBCs) remained normal throughout the study. This normal WBC count indicates the lack of injury, infection and/or inflammation in the eye. Furthermore, chemical analysis showed that the amounts of material dissolved in the fluid remained normal. No evidence of infection or the appearance of infectious agents was observed in any treated eye at any during the study.

These results demonstrate that the ATRIGEL® Delivery System is well tolerated and appears to be biologically inert following injection into and around the eye. In fact, ATRIGEL® drug products will reduce the toxic affects of certain drugs. For example, in a follow-on study, a formulation prepared by mixing the ATRIGEL® Delivery System with a compound that produces irritation in the eye was compared to the affect of the same material dissolved in water. Direct observations showed that the irritant dissolved in water produced significant swelling, severe redness and a watery discharge from the eye. In addition, the covering over the front part of the eye (the cornea) changed from transparent to cloudy. This change in the cornea resulted in the partial or complete loss of vision. However, injection of the ATRIGEL® Delivery System containing the irritant showed only mild to moderate swelling, moderate redness and the covering over the eye remained clear. This reduction in irritation is attributed to the ATRIGEL® Delivery System slowly releasing the irritant into the eye over a long period as compared to instantaneous exposure of the eye to high concentrations of the irritant from the water solution. This slow release reduces the toxic affect of the irritant and minimizes the possibility for permanent damage.

In conclusion, ATRIGEL® formulations containing PEG300, mPEG350 and NMP are acceptable vehicles for intravitreal or subconjuctival implantation.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

It is appreciated that certain features of the invention, which are, for clarity, described in the context of separate embodiments, may also be provided in combination in a single embodiment. Conversely, various features of the invention which are for brevity, described in the context of a single embodiment, may also be provided separately or in any sub-combination.

1. A flowable composition suitable for use as a controlled release implant, the composition comprising:
(a) a biodegradable, biocompatible thermoplastic polymer that is at least substantially insoluble in aqueous medium, water or body fluid;
(b) a biological agent, a metabolite thereof, a biological agently acceptable salt thereof, or a prodrug thereof; and
(c) a biocompatible organic liquid, at standard temperature and pressure, in which the thermoplastic polymer is soluble;
wherein the composition is suitable for ocular delivery.

2. The composition of claim 1 wherein the biodegradable, biocompatible thermoplastic polymer is a linear polymer.

3. The composition of claim 1 wherein the biodegradable, biocompatible thermoplastic polymer is a branched polymer.

4. The composition of claim 1 wherein the biodegradable, biocompatible thermoplastic polymer has a formula incorporating monomeric units selected from the group of lactides, glycolides, caprolactones, glycerides, anhydrides, amides, urethanes, esteramides, orthoesters, dioxanones, acetals, ketals, carbonates, phosphazenes, hydroxybutyrates, hydroxyvalerates, alkylene oxalates, alkylene succinates, amino acids, and any combination thereof; and the formula contains the monomeric units random or block order.

5. The composition of claim 1 wherein the biodegradable, biocompatible thermoplastic polymer is a polymer or copolymer of lactide monomeric units, caprolactone monomeric units, glycolide monomeric units, or any combination thereof.

6. The composition of claim 1 wherein the biodegradable, biocompatible thermoplastic polymer comprises a polymer selected from the group of polylactides, polyglycolides, polycaprolactones, polydioxanones, polycarbonates, polyhydroxybutyrates, polyalkyene oxalates, polyanhydrides, polyamides, polyesteramides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, poly(methyl vinyl ether), polyesters, polyalkylglycols, copolymers thereof, block copolymers thereof, terpolymers thereof, combinations thereof, and mixtures thereof.

7. The composition of claim 1 wherein the biodegradable, biocompatible thermoplastic polymer comprises at least one polyester.

8. The composition of claim 1 wherein the biodegradable, biocompatible thermoplastic polymer is at least one of a polylactide, a polyglycolide, a polycaprolactone, a copolymer thereof, a terpolymer thereof, or any combination thereof.

9. The composition of claim 1 wherein the biodegradable, biocompatible thermoplastic polymer is a poly(DL-lactide-co-glycolide).

10. The composition of claim 1 wherein the biodegradable, biocompatible thermoplastic polymer is a poly(DL-lactide-co-glycolide) having a carboxy terminal group.

11. The composition of claim 1 wherein the biodegradable, biocompatible thermoplastic polymer is a poly(DL-lactide-co-glycolide) without a carboxy terminal group.

12. The composition of claim 1 wherein the biodegradable, biocompatible thermoplastic polymer is 50/50 poly(DL-lactide-co-glycolide) having a carboxy terminal group.

13. The composition of claim 1 wherein the biodegradable, biocompatible thermoplastic polymer is 75/25 poly(DL-lactide-co-glycolide) without a carboxy terminal group.

14. The composition of claim 1 wherein the biodegradable, biocompatible thermoplastic polymer is present in up to about 80 wt. % of the composition.

15. The composition of claim 1 wherein the biodegradable, biocompatible thermoplastic polymer is present in more than about 10 wt. % of the composition.

16. The composition of claim 1 wherein the biodegradable, biocompatible thermoplastic polymer is present in about 10 wt. % to about 80 wt. % of the composition.

17. The composition of claim 1 wherein the biodegradable, biocompatible thermoplastic polymer is present in about 30 wt. % to about 50 wt. % of the composition.

18. The composition of claim 1 wherein the biodegradable, biocompatible thermoplastic polymer has an average molecular weight of more than about 15,000.

19. The composition of claim 1 wherein the biodegradable, biocompatible thermoplastic polymer has an average molecular weight of up to about 45,000.

20. The composition of claim 1 wherein the biodegradable, biocompatible thermoplastic polymer has an average molecular weight of about 15,000 to about 45,000.

21. The composition of claim 1 wherein the biocompatible organic liquid has a water solubility ranging from completely insoluble in any proportion to completely soluble in all proportions.

22. The composition of claim 1 wherein the biocompatible organic liquid is completely insoluble in water but will diffuse into body fluid.

23. The composition of claim 1 wherein the biocompatible organic liquid is at least partially water-soluble.

24. The composition of claim 1 wherein the biocompatible organic liquid is completely water-soluble.

25. The composition of claim 1 wherein the biocompatible organic liquid is a polar protic liquid.

26. The composition of claim 1 wherein the biocompatible organic liquid is a polar aprotic liquid.

27. The composition of claim 1 wherein the biocompatible organic liquid is a cyclic, aliphatic, linear aliphatic, branched aliphatic or aromatic organic compound, that is liquid at ambient and physiological temperature, and contains at least one functional group selected from the group of alcohols, ketones, ethers, amides, amines, alkylamines, esters, carbonates, sulfoxides, sulfones, and sulfonates.

28. The composition of claim 1 wherein the biocompatible organic liquid is selected from the group of substituted heterocyclic compounds, esters of carbonic acid and alkyl alcohols, alkyl esters of monocarboxylic acids, aryl esters of monocarboxylic acids, aralkyl esters of monocarboxylic acids, alkyl esters of dicarboxylic acids, aryl esters of dicarboxylic acids, aralkyl esters of dicarboxylic acids, alkyl esters of tricarboxylic acids, aryl esters of tricarboxylic acids, aralkyl esters of tricarboxylic acids, alkyl ketones, aryl ketones, aralkyl ketones, alcohols, polyalcohols, alkylamides, dialkylamides, alkylsulfoxides, dialkylsulfoxides, alkylsulfones, dialkylsulfones, lactones, cyclic alkyl amides, cyclic alkyl amines, aromatic amides, aromatic amines, mixtures thereof, and combinations thereof.

29. The composition of claim 1 wherein the biocompatible organic liquid is selected from the group of N-methyl-2-pyrrolidone, 2-pyrrolidone, (C₂-C₈)aliphatic alcohol, glycerol, tetraglycol, glycerol formal, 2,2-dimethyl-1,3-dioxolone-4-methanol, ethyl acetate, ethyl lactate, ethyl butyrate, dibutyl malonate, tributyl citrate, tri-n-hexyl acetylcitrate, diethyl succinate, diethyl glutarate, diethyl malonate, triethyl citrate, triacetin, tributyrin, diethyl carbonate, propylene carbonate, acetone, methyl ethyl ketone, dimethylacetamide, dimethylformamide, caprolactam, dimethyl sulfoxide, dimethyl sulfone, tetrahydrofuran, caprolactam, N,N-diethyl-m-toluamide, 1-dodecylazacycloheptan-2-one, 1,3-dimethyl-3,4,5,6-tetrahydro-2-(1H)-pyrimidinone, benzyl benzoate, and combinations thereof.

30. The composition of claim 1 wherein the biocompatible organic liquid has a molecular weight in the range of about 30 to about 500.

31. The composition of claim 1 wherein the biocompatible organic liquid is N-methyl-2-pyrrolidone, 2-pyrrolidone, N,N-dimethylformamide, dimethyl sulfoxide, propylene carbonate, caprolactam, triacetin, or any combination thereof.

32. The composition of claim 1 wherein the biocompatible organic liquid is N-methyl-2-pyrrolidone.

33. The composition of claim 1 wherein the biocompatible liquid is present in more than about 40 wt. % of the composition.

34. The composition of claim 1 wherein the biocompatible liquid is present in up to about 80 wt. % of the composition.

35. The composition of claim 1 wherein the biocompatible liquid is present in about 50 wt. % to about 70 wt. % of the composition.

36. The composition of claim 1 wherein the biocompatible liquid is dispersible in aqueous medium, water, or body fluid.

37. The composition of claim 1 wherein the biological agent is independently selected from the group of adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunt to; anesthetic; anorectic; antagonist; anterior pituitary suppressant; anthelmintic; antiacne agent; anti-adrenergic; anti-allergic; anti-amebic; anti-androgen; anti-anemic antianginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiurietic; antidote; anti-emetic; anti-epileptic; anti-estrogen; antifibronolytic; antifungal; antiglaucoma agent; antihemophilic; antihermorrhagic; antihistamine; antihyperlipidemia; antihyperlipoproteinemic; antihypertensive; antihypotensive; anti-infctive; anti-infective, topical; anti-inflammatory; antikeratinizing agent; antimalarial; antimicrobial; antimigraine; antimycotic, antinausant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergie diagnostic aid; diuretic; dopaminergic agent; ectoparasiticide; emetic; enxzyme inhibitor; estrogen; fibrinolytic; flourescent agent; free oxygen radical scavenger; gastrointestinal motility effector; glucocorticoid; gonad-stimulating principle; hair growth stimulant; hemostatic; histamine H2 receptor antagonist; hormone; hypocholesterolemic; hypoglycemic; hypolipidemic; hypotensive; imaging agent; immunizing agent; immunomodulator; immunoregulator; immunostimulant; immunosuppressant; impotence therapy; inhibitor; keratolytic; LNRN agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregaton inhibitor; post-stroke and post-head trauma treatment; potentiator; progestin; prostaglandin; prostate growth inhibitor; prothyrotropin; psychotropic; radioactive agent; regulator; relaxant; repartitioning agent; scabicide; sclerosing agent; sedative; sedative-hypnotic; selective adenosine A1 antagonist; serotonin antagonist; serotinin inhibitor; serotinin receptor antagonist; steroid; stimulant; suppressant; symptomatic multiple sclerosis; synergist; thyroid hormone; thyroid inhibitor; thyromimetic; tranquilizer; treatment of amyotrophic laterial sclerosis; treatment of cerebral ischemia; treatment of Paget's disease; treatment of unstable angina; uricosuric; vasoconstrictor; vasodilator; vulnerary; wound healing agent; zxanthine oxidase inhibitor; and combinations thereof.

38. The composition of claim 1 wherein the biological agent is independently selected from the group of Acebutolol; Acebutolol; Acyclovir; Albuterol; Alfentanil; Almotriptan; Alprazlam; Amiodarone; Amlexanox; Amphotericin B; Atorvastatin; Atropine; Auranofin; Aurothioglucose; Benazepril; Bicalutamide; Bretylium; Brifentanil; Bromocriptine; Buprenorphine; Butorphanol; Buspirone; Calcitonin; Candesartan; Carfentanil; Carvedilol; Chlorpheniramine; Chlorothiazide; Chlorphentermine; Chlorpromazine; Clindamycin; Clonidine; Codeine; Cyclosporine; Desipramine; Desmopressin; Dexamethasone; Diazepam; Diclofenac; Digoxin; Digydrocodeine; Dolasetron; Dopamine; Doxepin; Doxycycline; Dronabinol; Droperidol; Dyclonine; Eletriptan; Enalapril; Enoxaparin; Ephedrine; Epinephrine; Ergotamine; Etomidate; Famotidine; Felodipine; Fentanyl; Fexofenadine; Fluconazole; Fluoxetine; Fluphenazine; Flurbiprofen; Fluvastatin; Fluvoxamine; Frovatriptan; Furosemide; Ganciclovir; Gold sodium thiomalate; Granisetron; Griseofulvin; Haloperidol; Hepatitis B Virus Vaccine; Hydralazine; Hydromorphone; Insulin; Ipratropium; Isradipine; Isosorbide Dinitrate; Ketamine; Ketorolac; Labetalol; Levorphanol; Lisinopril; Loratadine; Lorazepam; Losartan; Lovastatin; Melatonin; Methyldopa; Methylphenidate; Metoprolol; Midazolam; Mirtazapine; Morhpine; Nadolol; Nalbuphine; Naloxone; Naltrexone; Naratriptan; Neostgmine; Nicardipine; Nifedipine; Norepinephrine; Nortriptyline; Octreotide; Olanzapine; Omeprazole; Ondansetron; Oxybutynin; Oxycodone; Oxymorphone; Oxytocin; Phenylephrine; Phenylpropanolaimine; Phenytoin; Pimozide; Pioglitazone; Piroxicam; Pravastatin; Prazosin; Prochlorperazine; Propafenone; Prochlorperazine; Propiomazine; Propofol; Propranolol; Pseudoephedrine; Pyridostigmine; Quetiapine; Raloxifene; Remifentanil; Rofecoxib; repaglinide; Risperidone; Rizatriptan; Ropinirole; Scopolamine; Selegiline; Sertraline; Sildenafil; Simvastatin; Sirolimus; Spironolactone; Sufentanil; Sumatriptan; Tacrolimus; Tamoxifen; Terbinafine; Terbutaline; Testosterone; Tetanus toxoid; THC Tolterodine; Triamterene; Triazolam; Tricetamide; Valsartan; Venlafaxine; Verapamil; Zaleplon; Zanamivir; Zafirlukast; Zolmitriptan; Zolpidem; and combinations thereof.

39. The composition of claim 1 wherein the cell-cycle biological agent, schedule-dependant biological agent, metabolite thereof, biological agently acceptable salt thereof, or prodrug thereof is present in more than about 0.00001 wt. % of the composition.

40. The composition of claim 1 wherein the cell-cycle biological agent, schedule-dependant biological agent, metabolite thereof, biological agently acceptable salt thereof, or prodrug thereof is present in up to about 20 wt. % of the composition.

41. The composition of claim 1 wherein the cell-cycle biological agent, schedule-dependant biological agent, metabolite thereof, biological agently acceptable salt thereof, or prodrug thereof is present in about 0.00001 wt. % to about 10 wt. % of the composition.

42. The composition of claim 1 wherein the human maximum tolerated dose (MTD) of the cell-cycle biological agent, schedule-dependant biological agent, metabolite thereof, or prodrug thereof, present in the flowable composition is less than the human maximum tolerated dose (MTD) of the cell-cycle biological agent, schedule-dependant biological agent, metabolite thereof, or prodrug thereof, present in solution.

43. The composition of claim 1 wherein the human maximum tolerated dose (MTD) of the cell-cycle biological agent, schedule-dependant biological agent, metabolite thereof, or prodrug thereof, present in the flowable composition is at least 50% less than the human maximum tolerated dose (MTD) of the cell-cycle biological agent, schedule-dependant biological agent, metabolite thereof, or prodrug thereof, present in solution.

44. The composition of claim 1 further comprising at least one of:
a release rate modification agent for controlling the rate of release of the biological agent in vivo from an implant matrix;
a pore-forming agent;
a biodegradable, crystallization-controlling agent;
a plasticizer;
a leaching agent;
a penetration enhancer;
an absorption altering agent;
an opacification agent; and
a colorant.

45. The composition of claim 44 wherein the release rate modification agent is selected from the group of an ester of a monocarboxylic acid, an ester of a dicarboxylic acid, an ester of a tricarboxylic acid, a polyhydroxy alcohol, a fatty acid, a triester of glycerol, a sterol, an alcohol, and any combination thereof.

46. The composition of claim 44 wherein the release rate modification agent is selected from the group of 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, propylene glycol, polyethylene glycol, glycerin, sorbitol, triglyceride, epoxidized soybean oil, cholesterol, a (C₆-C₁₂)alkanol, 2-ethoxyethanol, and any combination thereof.

47. The composition of claim 44 wherein the pore-forming agent is a sugar, salt, water-soluble polymer, or water-soluble organic liquid.

48. The composition of claim 44 wherein the biodegradable, crystallization-controlling agent is selected from the group of calcium carbonate, hydroxyapatite, calcium phosphate, calcium apatite, calcium sulfate, calcium bicarbonate, calcium chloride, sodium carbonate, sodium bicarbonate, sodium chloride, calcium stearate, calcium palmitate, sodium stearate, dextran, starch, sodium carboxymethyl cellulose, carboxymethyl cellulose, hydroxyethyl cellulose, hydroxypropyl cellulose, cross-linked sodium carboxymethyl cellulose, poly(vinyl alcohol), glycerol palmitate, glycerol stearate, triethyl citrate, ethyl lactate, poly(ethylene glycol), poly(vinyl pyrrolidone), poly(lactide-co-caprolactone), and combinations thereof.

49. The composition of claim 44 wherein the modifying agent is selected from the group of benzyl benzoate, phthalic esters, benzylphthalates, glycol benzoates, trimellitates, adipates, azelates, sebacates, esters of aliphatic and aromatic di- and tricarboxylic acids, organic phosphates, sesame oil, soybean oil, cotton seed oil, almond oil, sunflower oil, peanut oil, and combinations thereof.

50. The composition of claim 44 wherein the absorption altering agent is selected from the group of propylene glycol, glycerol, urea, diethyl sebecate sodium, lauryl sulfate, sodium lauryl sulfate, sorbitan ethoxylates, oleic acid, pyrrolidone carboxylate esters, N-methylpyrrolidone, N,N-diethyl-m-tolumide, dimethyl sulfoxide, alkyl methyl sulfoxides, and combinations thereof.

51. The composition of claim 44 wherein the rate modification agent is a water insoluble organic substance.

52. The composition of claim 51 wherein the water insoluble organic substance is an ester of a mono-, di- or tricarboxylic acid.

53. The composition of claim 44 wherein the opacification agent comprises barium, iodine, or calcium.

54. The composition of claim 1 wherein the biological agent, metabolite thereof, biological agently acceptable salt thereof, or prodrug thereof is incorporated into a particulate or encapsulated controlled-release component.

55. The composition of claim 54 wherein the particulate controlled-release component comprises a conjugate in which the biological agent, metabolite thereof, biological agently acceptable salt thereof, or prodrug thereof is covalently bonded to a carrier molecule.

56. The composition of claim 54 wherein the particulate controlled-release component is a microstructure selected from the group of a microcapsule, a nanoparticle, a cyclodextrin, a liposome, and a micelle.

57. The composition of claim 54 wherein the particulate controlled-release component is a microstructure of less than about 500 microns.

58. The composition of claim 54 wherein the particulate controlled-release component is a macrostructure selected from the group of a fiber, film, rod, disc and cylinder.

59. The composition of claim 54 wherein the particulate controlled release-component is a macrostructure of at least about 500 microns.

60. The composition of claim 1 that is capable of forming a solid microporous matrix, the matrix being a core surrounded by a skin and the core containing pores of diameters from about 1 to about 1000 microns.

61. The composition of claim 60 wherein the skin contains pores of smaller diameters than those of the core pores such that the skin is functionally non-porous in comparison with the core.

62. The composition of claim 1 having a volume of more than about 0.001 mL.

63. The composition of claim 1 having a volume of up to about 20.0 mL.

64. The composition of claim 1 having a volume of about 0.01 mL to about 10.0 mL.

65. The composition of claim 1 that is formulated for administration less than about once per week.

66. The composition of claim 1 that is formulated for administration more than about once per year.

67. The composition of claim 1 that is formulated for administration about once per week to about once per year.

68. The composition of claim 1 that delivers the biological agent, metabolite thereof, biological agently acceptable salt thereof, or prodrug thereof to mammalian tissue at a dosage of about 1 picogram/kilogram/day to about 1 milligram/kilogram/day.

69. The composition of claim 68 wherein the delivery is systemic delivery.

70. The composition of claim 68 wherein the delivery is local delivery.

71. The composition of claim 68 wherein the dosage is delivered locally for a period of time of up to about 1 year.

72. The composition of claim 68 wherein the dosage is delivered locally for a period of time of up to about 1 month.

73. The composition of claim 68 wherein the dosage is delivered locally for a period of time of up to about 1 week.

74. The composition of claim 68 wherein the dosage is delivered locally for a period of time of more than about 1 day.

75. The composition of claim 1 further comprising a second biological agent.

76. A method of treating a disease or disorder in a mammal, the method comprising administering to the ocular region of a mammal in need of such treatment an effective amount of the flowable composition of claim 1.

77. The method of claim 76 wherein the mammal is a human.

78. The method of claim 76 wherein the flowable composition is administered in multiple locations of the ocular region of the mammal.

79. A method for locally delivering a biological agent via the ocular region of a mammal, the method comprising contacting the ocular region of the mammal with the flowable composition of claim 1.

80. A method for systemically delivering a biological agent via an ocular region of a mammal, the method comprising contacting the ocular region of the mammal with the flowable composition of claim 1.

81. An implant comprising:
(a) a biodegradable, biocompatible thermoplastic polymer that is at least substantially insoluble in aqueous medium, water or body fluid;
(b) a biological agent, a metabolite thereof, a biological agently acceptable salt thereof, or a prodrug thereof; and
(c) a biocompatible organic liquid at standard temperature and pressure, in which the thermoplastic polymer is soluble;
wherein the implant is located in the ocular region of a mammal and the implant has a solid or gelatinous microporous matrix, the matrix being a core surrounded by a skin and wherein the implant is surrounded by body tissue.

82. The implant of claim 81 that has fully coagulated.

83. The implant of claim 81 that has solidified

84. The implant of claim 81 wherein the amount of biocompatible organic liquid decreases over time.

85. The implant of claim 81 wherein the core contains pores of diameters from about 1 to about 1000 microns.

86. The implant of claim 81 wherein the skin contains pores of smaller diameters than those of the core pores.

87. The implant of claim 81 wherein the skin pores are a size such that the skin is functionally non-porous in comparison with the core.

88. An implant comprising:
(a) a biodegradable, biocompatible thermoplastic polymer that is at least substantially insoluble in aqueous medium, water or body fluid; and
(b) a biological agent, a metabolite thereof, a biological agently acceptable salt thereof, or a prodrug thereof;
wherein the implant is located in the ocular region of a mammal and the implant has a solid or gelatinous microporous matrix, the matrix being a core surrounded by a skin and wherein the implant is surrounded by body tissue.

89. The implant of claim 88 wherein the core contains pores of diameters from about 1 to about 1000 microns.

90. The implant of claim 88 wherein the skin contains pores of smaller diameters than those of the core pores.

91. The implant of claim 88 wherein the skin pores are a size such that the skin is functionally non-porous in comparison with the core.

92. A method of forming an implant in situ within the ocular region of a living body, the method comprising:
(a) injecting a flowable composition within the ocular region of a patient, the flowable composition of claim 1; and
(b) allowing the biocompatible organic liquid to dissipate to produce a solid biodegradable implant.

93. A biological agent kit suitable for in situ formation of a biodegradable implant in an ocular region, the kit comprising:
(a) a first container comprising a flowable composition suitable for delivery into an ocular region, the composition comprising:
(i) a biodegradable, biocompatible thermoplastic polymer that is at least substantially insoluble in aqueous medium, water or body fluid; and
(ii) a biocompatible organic liquid at standard temperature and pressure, in which the thermoplastic polymer is soluble;
(b) a second container comprising a biological agent, a metabolite thereof, a biological agently acceptable salt thereof, or a prodrug thereof.

94. The kit of claim 93 wherein the first container is a syringe.

95. The kit of claim 93 wherein the second container is a syringe.

96. The kit of claim 93 wherein the first container is a syringe, the second container is a syringe, and both syringes are configured to directly connect to each other.

97. The kit of claim 93 further comprising instructions.

98. A method of treating a disease or disorder associated with the ocular region of a mammal, the method comprising administering to the ocular region of a mammal in need of such treatment an effective amount of the flowable composition of any one of claims 1-75.

99. The method of claim 98, wherein the disease or disorder associated with the ocular region is macular degeneration.

100. The method of claim 98, wherein the disease or disorder associated with the ocular region is cancer.

101. The method of claim 76, wherein the route of administration is intravitreal injection.

102. The method of claim 76, wherein the route of administration is subconjunctival injection

103. The method of claim 76, wherein the route of administration is subtenon injection

104. The method of claim 76, wherein the route of administration is intraocular injection

105. The method of claim 76, wherein the route of administration is retrobulbar injection.

106. The method of claim 76, wherein less than about 750 μL of the flowable composition is administered.

107. The method of claim 76, wherein less than about 500 μL of the flowable composition is administered.

108. The method of claim 76, wherein less than about 250 μL of the flowable composition is administered.

109. The method of claim 76, wherein about 10 μL to about 200 μL of the flowable composition is administered.

110. The method of claim 76, wherein about 5 μL to about 100 μL of the flowable composition is administered.
